SEQUENCE LISTING (30 sequences)

SEQ ID NO: 1 - protein, 122 aa, Artificial Sequence
  Synthetic
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAA

SEQ ID NO: 2 - protein, 329 aa, Artificial Sequence
  Synthetic
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 329 aa, Artificial Sequence
  Synthetic
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 468 aa, Artificial Sequence
  Synthetic
MMRPIVLVLLFATSALAQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 468 aa, Artificial Sequence
  Synthetic
MMRPIVLVLLFATSALAQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 451 aa, Artificial Sequence
  Synthetic
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 451 aa, Artificial Sequence
  Synthetic
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 106 aa, Artificial Sequence [sequence omitted]

SEQ ID NO: 9 - protein, 107 aa, Artificial Sequence [sequence omitted]

SEQ ID NO: 10 - protein, 11 aa, Artificial Sequence
  Synthetic
APTSSSGGGGS

SEQ ID NO: 11 - protein, 180 aa, Artificial Sequence
  Synthetic
EPHSLSYDITVIPKFRPGPRWCAVQGQVDEKTFLHYDCGQKTVTPVSPLGKKLQVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 12 - protein, 421 aa, Artificial Sequence
  Synthetic
MMRPIVLVLLFATSALAQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGQKTVTPVSPLGKKLQVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 13 - protein, 404 aa, Artificial Sequence
  Synthetic
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGQKTVTPVSPLGKKLQVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 14 - protein, 4 aa, Artificial Sequence
  Synthetic
GGGS

SEQ ID NO: 15 - protein, 135 aa, Artificial Sequence
  Synthetic
LFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTV

SEQ ID NO: 16 - protein, 71 aa, Artificial Sequence
  Synthetic
TTTPAPRPPTPAPTIASQPLSLRPEACRPAAGGAVHTRGLDFACDIYIWAPLAGTCGVLLLSLVITLYCSL

SEQ ID NO: 17 - protein, 42 aa, Artificial Sequence
  Synthetic
KRGRKKLLYIFKQPFMRPVQTTQEEDGCSCRFPEEEEGGCEL

SEQ ID NO: 18 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 360 aa, Artificial Sequence
  Synthetic
LFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 629 aa, Artificial Sequence
  Synthetic
MALPVTALLLPLALLLHAARPLFNQEVQIPLTESYCGPCPKNWICYKNNCYQFFDESKNWYESQASCMSQNASLLKVYSKEDQDLLKLVKSAHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCSLKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGSGSGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 21 - protein, 213 aa, Artificial Sequence
  Synthetic
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 210 bp, Artificial Sequence
  Synthetic
atgatgcggccaattgtcctcgtgctccttttcgccacctccgccctcgctcaagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttaccca

SEQ ID NO: 23 - DNA, 363 bp, Artificial Sequence
  Synthetic
caagttcagcttcagcagccgggggctgagttggtgaaacccggggccagtgtgaagatgagctgtaaagcgagcggctacaccttcacttcttataatatgcattgggttaagcaaacgccaggaagggggctggagtggatcggcgctatttacccaggtaacggtgacacatcatataaccaaaagtttaagggaaaggcaaccctcacagcggacaagagtagctcaaccgcatacatgcaactgtcaagccttacctccgaagacagcgcagtgtactactgcgccagaagcacctactatgggggtgattggtacttcaacgtctggggggctggcaccacagtgactgtaagcgca

SEQ ID NO: 24 - DNA, 990 bp, Artificial Sequence
  Synthetic
gcgtcgaccaagggcccgtcagtgttcccgctggccccgtcatccaagtccacgtctgggggcacagcagccctgggatgcttggtcaaggactacttccccgagcccgtgactgtgtcctggaactccggagcactgacctccggagtgcacacctttcccgcggtgctgcagtcctccggactgtactccctgtcgtcggtcgtgaccgtgccgagctcctcgctcggaacccagacctacatctgcaacgtgaaccacaagccctcgaacaccaaagtggacaagaaggtcgagcccaaaagctgcgacaagactcacacttgtccgccgtgccccgcccccgaactgctgggtggcccctccgtgttcctgttcccgcctaagcctaaggacacccttatgatcagccgcacccctgaagtgacctgtgtcgtcgtggcagtgtcacacgaggacccggaggtcaagttcaattggtacgtggacggcgtggaagtgcataacgcaaagaccaagcctcgggaggaacagtacgcctcgacctaccgcgtggtgtcagtcctgactgtgctgcaccaggactggctgaacgggaaggagtacaagtgcaaagtgtcgaacaaggccctgccggctccaattgaaaagaccatcagcaaggccaagggccagccaagggaaccacaggtgtacaccctccctccttcccgggacgagctgaccaaaaaccaagtgtccctgacttgccttgtgaaggggttctacccttctgacattgccgtcgaatgggaatcgaacggacagcctgaaaacaactataagactaccccgcccgtgctggattccgacggaagcttcttcctgtactccaagctgaccgtggacaagtcgagatggcagcagggaaatgtgttcagctgctccgtgatgcatgaggcgctgcacaaccactacacccagaagtcactgagcctctcccccggaaag

SEQ ID NO: 25 - DNA, 1269 bp, Artificial Sequence
  Synthetic
atgatgcgccccatcgtgctcgtgctccttttcgctacctccgccctcgcccagatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaagcggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgcgcacctacctcaagctctggaggaggtggcagcgagccccatagtctgagctacgacatcacagttattcccaagttcaggcccggaccgcgctggtgtgccgtgcaaggacaagtcgacgaaaaaacctttcttcattacgattgcggacagaagactgtaacgccagtctctcctttaggtaagaagttacaggtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcgtagtga

SEQ ID NO: 26 - DNA, 317 bp, Artificial Sequence
  Synthetic
agatcgtgttgtcccaatcacccgcaattctctctgcgagcccaggggagaaggtgaccatgacttgccgcgcttcaagttccgtatcctacattcactggttccagcagaagcccggaagttcccctaagccctggatctatgctacatccaatctggcaagcggtgttcccgttagattttccggaagcgggtctggaaccagttacagtctgactatttccagggtcgaggccgaagatgcggctacttattattgccaacagtggacctctaacccacccacattcggcggcggcactaagttggaaattaag

SEQ ID NO: 27 - DNA, 321 bp, Artificial Sequence
  Synthetic
cggaccgtggccgccccgagcgtgttcattttccctccctccgacgagcagttgaaatcgggcaccgctagcgtggtctgccttctcaacaatttctatccacgggaagccaaagtgcagtggaaggtcgacaacgcgctccaatccgggaactcacaggaatccgtgactgagcaggattccaaggactcgacctactccctgtcatccacgctgaccctgagcaaggcagactacgagaagcacaaggtctacgcctgcgaagtgacacaccagggactgtccagccccgtgaccaagagcttcaacagaggagaatgc

SEQ ID NO: 28 - DNA, 540 bp, Artificial Sequence
  Synthetic
gagccccatagtctgagctacgacatcacagttattcccaagttcaggcccggaccgcgctggtgtgccgtgcaaggacaagtcgacgaaaaaacctttcttcattacgattgcggacagaagactgtaacgccagtctctcctttaggtaagaagttacaggtcactacggcgtggaaggcacaaaaccccgtcctgcgcgaggtcgtcgacatcctgactgaacaattgtgggacatccagctcgagaattacactccaaaggagcctcttaccctgcaggctagaatgtcttgcgagcaaaaggcagagggccactcctccggcagctggcagttcagtttcgacggacaaatctttctgttattcgattcagagaagagaatgtggactacagttcaccccggtgcccgtaaaatgaaggagaagtgggaaaacgacaaagtggtggcgactaagctttatctttggtcgatgggagactgcatcggttggctggaagatttcctcatgggtatggactccactttggagccatcg

SEQ ID NO: 29 - protein, 404 aa, Artificial Sequence
  Synthetic
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAPTSSSGGGGSEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPS

SEQ ID NO: 30 - protein, 179 aa, Artificial Sequence
  Synthetic
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEP

(19) United States
(12) Patent Application Publication
(10) Pub. No.: US 2024/0287202 A1
(43) Pub. Date: Aug. 29, 2024

(54) ANTIBODY-NKG2D LIGAND DOMAIN FUSION PROTEIN

(71) Applicant: XYPHOS BIOSCIENCES INC., South San Francisco, CA (US)

(72) Inventors: Kaman Kim, South San Francisco, CA (US); Kyle Landgraf, South San Francisco, CA (US)

(21) Appl. No.: 18/568,387

(22) PCT Filed: Jun. 8, 2021
(86) PCT No.: PCT/US21/36461

§ 371 Date: Dec. 8, 2023

(51) Int. Cl. C07K 16/28; A61K 39/00; A61P 35/00; C07K 14/705

(52) U.S. Cl. CPC C07K 16/2887; A61K 39/4611; A61K 39/4631; A61K 39/464424; A61P 35/00; C07K 14/7056; A61K 2039/505; A61K 2239/15; A61K 2239/39; C07K 2317/734; C07K 2317/92; C07K 2319/33

(57) ABSTRACT

The disclosure provides an antibody fusion protein comprising (i) heavy chains comprising variable region sequences comprising the amino acid sequence of SEQ ID NO: 1 and (ii) light chains comprising variable region sequences comprising the amino acid sequence of SEQ ID NO: 8, wherein the light chains are fused at the C-terminus to an A1-A2 domain comprising the amino acid sequence of SEQ ID NO: 11. Nucleic acids encoding all or part of the antibody fusion protein are provided, as well as methods of using the antibody fusion protein in the treatment of, e.g., CD20-positive cancers.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 59,942 byte ASCII (Text) file named “56867_Seqlisting.txt”; created on Jun. 8, 2021.

### FIELD OF THE INVENTION

The invention relates to antibodies fused to the A1-A2 domain of a non-natural NKG2D ligand that binds to non-natural NKG2D receptors.

### BACKGROUND

The engineering of patient-derived T cells to express chimeric antigen receptors (CARs) has altered the landscape of adoptive cell therapies, providing scientists and clinicians the ability to harness the powerful cytolytic capabilities of T cells and direct them to specific antigen-expressing targets in an MHC-independent manner. Their initial application to treat hematologic malignancies has led to astounding responses and inspired a surge of research efforts to drive their effective use in non-hematologic indications. However, CAR-T cell therapies are limited by their utilization of a single-purpose targeting domain, lack of dose control which can contribute to cytokine release syndrome, inability to address tumor antigen loss leading to disease relapse, and immunogenicity of non-human targeting domains leading to lack of persistence. There is a need in the art for improved CAR-based cell therapies to address these limitations of current therapy options.

### SUMMARY

The disclosure provides an antibody fusion protein comprising (i) heavy chains comprising variable region sequences comprising the amino acid sequence of SEQ ID NO: 1 and (ii) light chains comprising variable region sequences comprising the amino acid sequence of SEQ ID NO: 8, wherein the light chains are fused at the C-terminus to an A1-A2 domain comprising the amino acid sequence of SEQ ID NO: 11. In various aspects, the heavy chains comprise constant domains comprising the amino acid sequence of SEQ ID NO: 3. Optionally, the A1-A2 domain is fused to the light chains via a linker comprising the amino acid sequence of SEQ ID NO: 10. In this regard, the light chains, in various aspects, comprise the amino acid sequence of SEQ ID NO: 13. In various aspects, the heavy chains comprising the amino acid sequence of SEQ ID NO: 7.

The disclosure further provides a nucleic acid molecule comprising a nucleotide sequence encoding the light chain of the antibody fusion protein (e.g., a light chain comprising variable region sequence comprising the amino acid sequence of SEQ ID NO: 8, wherein the light chains are fused at the C-terminus to an A1-A2 domain comprising the amino acid sequence of SEQ ID NO: 11). The disclosure further provides a composition comprising the nucleic acid molecule encoding a light chain of the antibody fusion protein and a nucleic acid molecule comprising a nucleotide sequence encoding the heavy chain of the antibody fusion protein described herein (e.g., a heavy chain comprising a variable region sequence comprising the amino acid sequence of SEQ ID NO: 1). Also provided is an expression vector comprising the nucleic acid molecule encoding the light chain of the antibody fusion protein described herein, optionally further comprising a nucleic acid molecule comprising a nucleotide sequence encoding the heavy chain of the antibody fusion protein described herein. Further provided is a host cell comprising the expression vectors described herein. The disclosure provides a host cell comprising a nucleic acid molecule comprising a nucleotide sequence encoding the light chain of the antibody fusion protein and a nucleic acid molecule comprising a nucleotide sequence encoding the heavy chain of the antibody fusion protein. A method of producing an antibody fusion protein is also provided, the method comprising culturing a host cell comprising a nucleic acid molecule comprising a nucleotide sequence encoding the light chain of the antibody fusion protein and a nucleic acid molecule comprising a nucleotide sequence encoding the heavy chain of the antibody fusion protein, and recovering the antibody fusion protein.

Also provided is a kit comprising one or more containers comprising the antibody fusion protein described herein. Optionally, the kit further comprises one or more containers comprising a mammalian cell (e.g., human lymphocyte or a human macrophage) comprising a chimeric antigen receptor comprising SEQ ID NO: 15. In various aspects, the chimeric antigen receptor further comprises SEQ ID NOs: 16-18.

The disclosure further provides a method of treating a subject suffering from a CD20-positive cancer, the method comprising administering to the subject the antibody fusion protein described herein and a mammalian cell (e.g., human lymphocyte or a human macrophage) comprising a chimeric antigen receptor comprising SEQ ID NO: 15. Optionally, the chimeric antigen receptor further comprises SEQ ID NOs: 16-18. Use of the antibody fusion protein and mammalian cell to treat a CD20-positive cancer is provided, as well as use of the antibody fusion protein and mammalian cell in the preparation of medicaments to treat a CD20-positive cancer.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. As such, the terms “a” (or “an”), “one or more,” and “at least one” can be used interchangeably herein. The term “or” should be understood to encompass items in the alternative or together, unless context unambiguously requires otherwise.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. However, the description also contemplates the same ranges in which the lower and/or the higher endpoint is excluded. When the term “about” is used, it means the recited number plus or minus 5%, 10%, or more of that recited number. The actual variation intended is determinable from the context.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. Only such limitations which are described herein as critical to the invention should be viewed as such; variations of the invention lacking limitations which have not been described herein as critical are intended as aspects of the invention.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document.

### BRIEF DESCRIPTION OF THE DRAWINGS

FIG. 1 is a chart providing various sequences described herein.

FIG. 2A illustrates octet BLI kinetic binding data for His-tagged monomeric wild-type MIC ligand interaction with either wild-type NKG2D or INKG2D.YA. Fc-wtNKG2D or Fc-INKG2D.YA were captured with anti-human IgG Fc capture (AHC) biosensor tips associated with a dilution series of each ligand (parenthetical value indicates highest concentration examined) after baseline establishment. ULBP4 could not be expressed and purified as a monomer so was not included in this assay. Note that all axes are to the same scale (Binding-0 nm, 0.4 nm. 0.8 nm, 1.2 nm (y-axis); Time-0 s, 50 s, 100 s, 150 s, 200 s, 250 s, 300 s, 350 s (x-axis). Data is representative of a single experiment.

FIG. 2B illustrates results from ELISA confirming inability of iNKG2D.YA to engage natural ligands. Ligand-Fc fusions (R&D Biosystems) were coated onto microtiter plates and a titration of biotinylated Fc-wtNKG2D (dashed lines) or Fc-iNKG2D.YA (solid lines) applied and detected by streptavidin-HRP. (ELISA signal (OD450) (y-axis); nM Ligand Fc (x-axis).)

FIGS. 3A-3D: Orthogonal U2S3 ligand (A1-A2 domain) selective binding to NKG2D Y152A/199F (iNKG2D.AF) (an NKG2D ectodomain of the disclosure). Library design and phage panning performed was as described for iNKG2D. YA except that biotinylated double-mutant Fc-iNKG2D.AF was used during rounds of selection against increasing concentrations of Fc-wtNKG2D competitor. Data represents a single experiment (FIG. 3A) Octet BLI binding data for interaction of monomeric ligands to either Fc-wtNKG2D or Fc-INKG2D.AF. Data are representative of two experiments. (FIG. 3B) Lead variants selected from the phage display library were cloned as fusions to the C-terminus of the Rituximab light chain and differential binding to Fc-wtNKG2D, Fc-iNKG2D.YA, and Fc-iNKG2D.AF and quantified by ELISA. Shown are four variants that selectively engage Fc-INKG2D.YA and not the other two receptors. In the line graphs of FIG. 3B, wtNKG2D is represented by diamonds, iNKG2D is represented by squares, and iNKG2D.AF is represented by triangles. (FIG. 3C) ELISA demonstrating exclusivity of U2S3 and U2R ligand binding to the receptor variant against which it was selected—Fc-iNKG2D.YA and Fc-iNKG2D.AF, respectively. U2S3 ligands are further described in, e.g., U.S. Patent Publication No. 2019/0300594, hereby incorporated by reference. (FIG. 3D) Calcein release assay with Ramos target cells at an E:T of 20:1 with either INKG2D.YA-CAR or INKG2D.AF-CAR expressing CD8+ T cells and a titration of Rituximab.LC-U2S3 or Rituximab.LC-U2R. Error bars represent ±SD of technical replicates.

FIG. 4A: Relative binding of selected phage to Fc-iNKG2D.YA and Fc-wtNKG2D after the third and fourth rounds of panning in the presence of increasing concentrations of wtNKG2D competitor. Phage clones in the portion of the graph outlined by the red triangle were selected for further characterization.

FIG. 4B: Three phage variants-S1, S2, S3-were expressed as fusions to the C-terminus of the anti-FGFR3 antibody clone R3Mab heavy chain as MicAbodies and, along with wild-type ULBP2 and R81W versions, were tested for the ability of the selective variants to retain preferential Fc-INKG2D.YA binding (solid lines) over Fc-wtNKG2D (dashed lines). All purified MicAbodies retained binding to human FGFR3 (data not shown).

FIG. 4C: Binding analysis of His-tagged monomeric wild-type ULBP2, ULBP2 R81W, and the orthogonal U2S3 ligand binding to Fc-NKG2D and Fc-iNKG2D. YA. Fc-wtNKG2D or Fc-INKG2D. YA were captured with anti-human IgG Fc capture (AHC) biosensor tips then associated with a dilution series of ligand. Data are from single experiments.

FIG. 5: Octet BLI verification of U2S3 orthogonality when fused to the C-terminus of either the heavy or light chain of rituximab. Fc-wtNKG2D or Fc-iNKG2D.YA were captured with anti-human IgG Fc capture (AHC) biosensor tips then associated with a two-fold dilution series of MicAbody starting at 50 nM. The y-axes corresponding to binding responses were set to the same scale for all sensograms. Kd values could only be calculated for the two positive binding interactions are shown.

FIG. 6: Schematic of the iNKG2D. YA CAR receptor starting from the N-terminus of the polypeptide on the left and includes the signal sequence (SS) which is absent in the mature type I transmembrane protein. The underlined sequence corresponds to the signal sequence, the italicized sequence corresponds to the iNKG2D domain (SEQ ID NO: 15), the plain sequence corresponds to the CD8a hinge/transmembrane domain (SEQ ID NO: 16), the underlined and italicized sequence corresponds to the 4-1BB domain (SEQ ID NO: 17), the bolded sequence corresponds to the CD35 domain (SEQ ID NO: 18), the double underlined sequence corresponds to the linker, and the dotted underlined sequence corresponds to the eGFP (green fluorescent protein) sequence.

FIGS. 7A-7C: Elements of the convertibleCAR system. (FIG. 7A) Engineering overview to convert components of the NKG2D-MIC axis into the convertibleCAR system. INKG2D. YA and U2S3 became the components of a second generation CAR receptor and bispecific adaptor molecule (MicAbody), respectively. “TAA” is tumor-associated antigen. (FIG. 7B) Representative example of high efficiency lentiviral transduction of the iNKG2D. YA-CAR into either CD4 or CD8 cells. Transduction efficiency varied between donors but >70% GFP+ yields were consistently achieved. The RITscFv-CAR is shown for comparison and has the same architecture as the iNKG2D-CAR except that an scFv based upon the VH/VL domains of Rituximab was used instead of iNKG2D.YA. (FIG. 7C) Surface expression of INKG2D. YA-CAR was determined in CD8+ T cells by incubating cells with Rituximab.LC-U2S3 MicAbody followed by PE-conjugated mouse-anti-human kappa chain antibody staining. Untransduced T cells are shown for comparison.

FIGS. 8A-8C: Ligand-dependent activation of INKG2D-CAR expressing CD8+ T cells and MicAbody-dependent receptor internalization. (FIG. 8A) CD8+ T cells were transduced with CAR constructs comprised of either wild-type NKG2D or iNKG2D.YA as the receptor domain. Wild-type His-tagged monomeric ligands or His-tagged monomeric U2S3 were coated onto the wells of a microtiter plate in a 1:3 dilution series starting at 10 ug/mL. 1×10⁵ CAR expressing cells were introduced to the wells in 150 ul volume without exogenous IL2, supernatants collected 24 hours later, and the amount of cytokine produced and release quantified by cytokine-specific ELISA. ULBP4 was not included in the assay as a His-tagged version could not be expressed and purified. (FIG. 8B) CD8+ cells expressing either INKG2D-CAR or RITscFv-CAR were co-cultured with Ramos cells at an E:T of 4:1 with increasing concentrations of Rit-S3 MicAbody (nM) in the case of INKG2D-CAR cells. After 24 hours, culture supernatants were harvested and released cytokine quantified by ELISA. Cytolysis was measured by calcein release after two hours of co-incubation. All error bars are ±SD of technical triplicates. All data representative of a single experiment. (FIG. 8C) INKG2D-CD8+ cells were pre-incubated 5 nM Trastuzumab.LC-U2S3 MicAbody then exposed to wells pre-coated with a titration of Her2. After 2 hours, cells were incubated with anti-kappa-PE antibody to detect surface accessible MicAbody and GFP was examined to look for total levels of expressed INKG2D-CAR.

FIGS. 9A-9D: In vitro characterization of convertibleCAR activity. (FIG. 9A) Ramos (CD20+) target cells were exposed to convertibleCAR-CD8 cells at an E:T of 5:1 and co-cultured with increasing concentrations of Rituximab antibody (ADCC-deficient), Rituximab.LC-U2S3 MicAbody, or Trastuzumab.LC-U2S3 MicAbody. After 24 hours, supernatants were harvested and IL-2 (solid bars) or IFNγ (hatched bars) quantified by ELISA. Rit-U2S3 were the only samples that demonstrated a cytokine release at 5000 pg/ml or more. (FIG. 9B) ConvertibleCAR-CD8 cells were incubated with increasing concentrations of Alexa Fluor 647 conjugated Rituximab.LC-U2S3 for 30 minutes, the excess washed away, and the MFI quantified by flow cytometry. 5 nM indicates inflection point at which receptors are maximally occupied. (FIG. 9C) ConvertibleCAR-CD8 cells were armed with increasing concentrations of Rituximab.LC-U2S3 as described in (B) then co-incubated with calcein-loaded Ramos cells at an E:T of 20:1 for two hours after which the amount of released calcein was quantified. (FIG. 9D) iNKG2D.YA-CAR CD8+ cells were pre-armed with 5 nM Rituximab.LC-U2S3, 5 nM Trastuzumab.LC-U2S3, or an equimolar mixture of 2.5 nM of each as described in (B) then exposed to calcein-loaded Ramos or CT26-Her2 cells at two indicated E:T ratios. The amount of calcein released was quantified after two hours. Except for FIG. 9B, data are representative of at least two independent experiments and plotted as an average of technical triplicates.

FIGS. 10A-10C: Comparison of heavy-vs. light-chain U2S3 fusions to Rituximab (ADCC-) antibody. (FIG. 10A) Pharmacokinetics of serum Rituximab-U2S3 MicAbody levels after 100 ug IV administration in NSG mice in the absence of human T cells or tumor. All MicAbodies and antibody controls used were ADCC-deficient. The graph on the left is a comparison of parental antibody to the light-chain U2S3 fusion while the graph on the right is a comparison of parental antibody to the heavy-chain U2S3 fusion. All error bars are ±SD of technical triplicates. (FIG. 10B) In vitro calcein release assay after two hours co-culture with iNKG2D-CAR CD8+ T cells and Ramos target cells at an E:T of 20:1 and titrations of Rituximab-MicAbodies. Error bars represent ±SD for the experiment and data are representative of multiple experiments. The top line in the graph corresponds to Rituxumab.LC-U2S3, the middle line corresponds to Rituxumab.HC-U2S3, and the bottom line corresponds to Rituximab. (FIG. 10C) ELISA demonstrating binding of Rituximab.LC-U2S3 to mouse NKG2D. Shown are the A480 absorbance values. Trastuzumab.LC-Rae1b, with a mouse wild-type Rae1b ligand that binds naturally to mouse NKG2D, was included as a positive control.

FIGS. 11A-E: Control of a disseminated Raji B cell lymphoma in NSG mice. (FIG. 11A) Average luminescent output ±SD for each cohort along with individual animal traces for the groups that received (FIG. 11B) 5×10⁶ or (FIG. 11C) 15×10⁶ total T cells. T cell dynamics over the course of the study examining (FIG. 11D) human CD3+ cells in the blood and (FIG. 11E) bound MicAbody detected by anti-F(ab′)2. Shown are cohort averages ±SD, n=5.

FIGS. 12A-12C: Control of subcutaneously implanted Raji tumors in NSG mice by convertibleCAR-T cells. (FIG. 12A) Average tumor volumes for each cohort, n=5. Tumors varied greatly in size within each group so error bars were not graphed. Two cohorts, upside down triangles (7M cCAR-Ts+60 ug Ritux-S3) and squares (35M prearmed cCAR-Ts), overlap and cannot be graphically distinguished beyond day 26. (FIG. 12B) Bar graph illustrating Serum Rit-S3 levels at 14, 21, and 45 days post-implant. Three bars are provided for each time point-35M prearmed cCAR-Ts (left bar), 7M cCAR-Ts+60 ug Ritux-S3 (middle bar), and 35M cCAR-Ts+60 ug Ritux-S3 (right bar). Error bars indicated ±SD samples from mice in a given cohort. (FIG. 12C) CD3+ T cell dynamics in the blood and quantitation of the percentage of T cells with surface-associated MicAbody F(ab′)2 staining) with ±SD error bars shown.

FIGS. 13A-13F: Targeted recruitment of complement factor C1q to iNKG2D.AF-CAR cells to direct their complement-mediated attrition. (FIG. 13A) Structure of orthogonal ligand fusions to the Fc portion of human IgG expressed as either N- or C-terminal fusions. In addition to wild-type Fc, two sets of mutations in the CH2 domain that enhance C1q binding were independently explored-S267E/H268F/S324T/G236A/1332E (“EFTAE”) and K326A/E333A (“AA”). (FIG. 13B) ELISA examining binding of human C1q to each purified fusion protein. Rank order of Kd's was EFTAE<AA<wt (0.12, 0.35, and 0.67 nM, respectively) regardless of orientation of fusions. (FIGS. 13C and 13D) Complement-dependent cytotoxicity (CDC) assays for C1q-binding enhance Fc-fusions. INKG2D.AF-CAR or untransduced CD8+ T cells were incubated with a titration of each fusion molecule and 10% normal human serum complement for three hours before dead T cells were enumerated with SYTOX Red. (FIGS. 13E and 13F) CDC assays with U2S3 orthogonal ligand fusions to direct a complement to iNKG2D.YA-CAR cells as described in (FIG. 13C) above. All error bars are ±SD of triplicate technical measurements with the iNKG2D-AF and iNKG2D-YA performed as separate experiments.

FIGS. 14A-14E: Targeted delivery of mutant-IL2 cytokine to iNKG2D-CAR CD8+ T cells. (FIG. 14A) In vitro proliferation after three days of wtNKG2D-CAR (left bar) or iNKG2D.YA-CAR (right bar) treatment with 30 IUe/mL of cytokine or cytokine-U2S2 fusion. Darker shading is to highlight selectivity. (FIG. 14B) A low efficiency (45% GFP+) iNKG2D.YA-CAR transduction was cultured with 30 IUe/mL of non-selective (U2R81W) or iNKG2D.YA-selective (U2S2) mutIL2 fusion and maintained for seven days. Cells were periodically examined by flow cytometry to quantify the % GFP+ cells in each population. Top lines correspond to U2S2-hFc-mutIL2 (square) and U2S2-mutIL2 (circles); bottom lines correspond to U2R80W-mutIL2 (circles) and U2R80W-hFc-mutIL2 (squares). (FIG. 14C) iNKG2D-CAR CD8+ T cells were cultured with 30 IUe/mL of either wild-type IL-2 or U2S3-hFc-mutIL2 then co-cultured with Ramos cells at an E:T of 20:1 with increasing concentrations of Rituximab.LC-U2S3. Liberated calcein was quantified, and untransduced CD8+ cells were maintained in rhIL-2 served as a negative control. (FIG. 14D) Untransduced (right bar) or iNKG2D-CAR CAR CD8+ T cells (left bar) were incubated with various cytokine molecules for three-days and proliferation quantified. Control molecules included a monomeric U2S3-hFc as well as Rit-S3 MicAbody. Parenthetical values are IUe/mL concentrations tested. Data shown are an average of technical triplicates. (FIG. 14E) Serum PK of U2S3-hFc-mutIL2 after 60 ug IP injection in NSG mice (N=3). All error bars are ±SD of biological triplicates. Data are representative of at least two experiments.

FIGS. 15A-15B: In vivo response of convertibleCAR-T cells to U2S3-hFc-mutIL2. (FIG. 15A) NSG mice were injected with 7×10⁶ total iNKG2D-transduced cells (CD4:CD8 1:1). After contraction of T cells at 14 days, mice were injected with 30 ug U2S3-hFc-mutIL2 or PBS once per week (noted by triangles) and T cell dynamics monitored by flow cytometry. Shown are the % of human CD3+ T cells in the peripheral blood with each trace corresponding to an individual mouse, n=5. (FIG. 15B) Plots for the expansion of CD8+ cells as well as the increase in proportion of GFP+(CAR-expressing) cells upon U2S3-mutIL2 treatment. Upper cluster of lines corresponds to % GFP+ of CD8+ cells, lower cluster of lines corresponds to % CD8+ in blood.

FIG. 16: Responsiveness of human PBMCs to U2S3-hFc-mutIL2. Human PBMCs from three donors were incubated with increasing concentrations of U2S3-hFc-mutIL2 or U2S3-hFc-wtIL2 for four days along with controls. Each of the labeled cell types was examined for the marker Ki-67 to quantify proliferative response under each condition. Eleven bars are shown for each of donor 1, 2, and 3; the bars represent, from left to right in each panel, untreated, anti-CD3 [2 ug/ml], IL-2 [300 IUe/ml], mutIL2 [30 IUe/ml], mutIL2 [300 IUe/ml], mutIL2 [3000 IUe/ml], mutIL2 [30000 IUe/ml], wtIL2 [30 IUe/ml], wtIL2 [300 IUe/ml], wtIL2 [3000 IUe/ml], and wtIL2 [3000 IUe/ml]. Error bars are ±SD of triplicate measurements and data represents a single experiment.

FIGS. 17A-17B illustrate a study evaluating MicAbodies having the A1-A2 domain attached at different locations and using different linkers. FIG. 17A illustrates the constructs tested. Rit.HCd.S3 corresponds to Rituximab antibodies comprising a U2S3 A1-A2 domain, described in the Example fused to the heavy chains via a GGGS (SEQ ID NO: 14) linker. Rit.HCd.apts.S3 corresponds to Rituximab antibodies comprising a U2S3 A1-A2 domain fused to the heavy chains via a APTSSSGGGGS (SEQ ID NO: 10) linker. Rit.HCd.LC.S3 corresponds to Rituximab antibodies comprising a U2S3 A1-A2 domain fused to the light chains via a APTSSSGGGGS (SEQ ID NO: 10) linker. Rit.HCd.LC.gggs.S3 corresponds to Rituximab antibodies comprising a U2S3 A1-A2 domain fused to the light chains via a GGGS (SEQ ID NO: 14) linker. FIG. 17B is a bar graph illustrating cytolysis (% max; y-axis) achieved using various concentrations of the MicAbodies in an in vitro calcein release assay after two hours co-culture with INKG2D-CAR CD8+ T cells and Ramos target cells at an E:T of 20:1 and titrations of Rituximab-MicAbodies. For each construct, % max of cytolysis is illustrated for 0 nM (first bar), 0.008 nM (second bar), 0.04 nM (third bar), 0.2 nM (fourth bar), 1 nM (fifth bar), and 5 nM (sixth bar) MicAbody. Rit.HCd.LC.S3 (comprising the A1-A2 domain on the light chains linked by the APTSSSGGGGS (SEQ ID NO: 10) linker) performed better than the version with the GGGS (SEQ ID NO: 14) linker and better than the constructs having the A1-A2 domain fused to the heavy chains, regardless of linker.

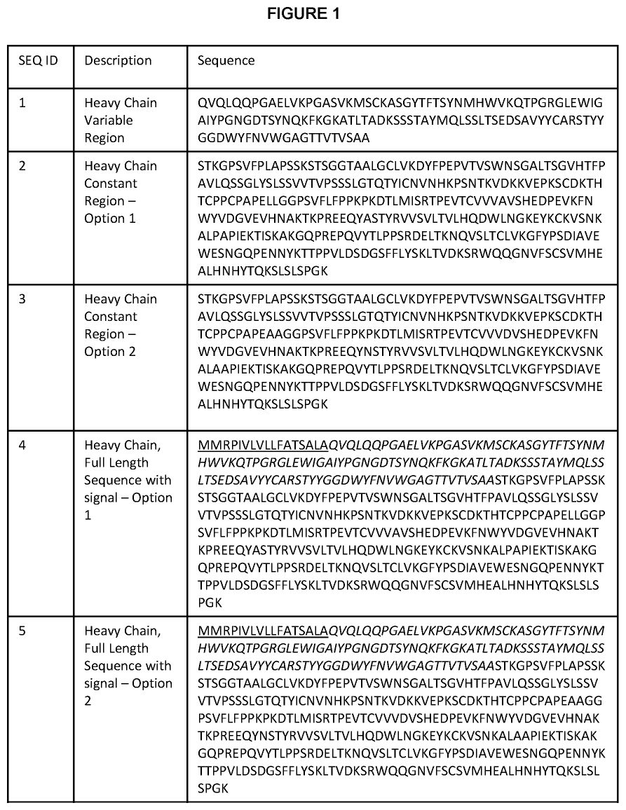

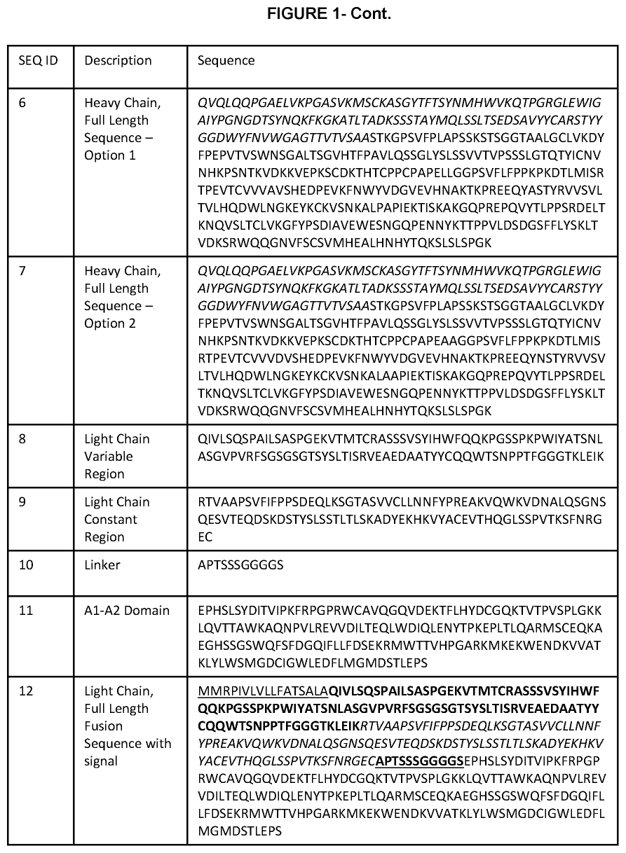

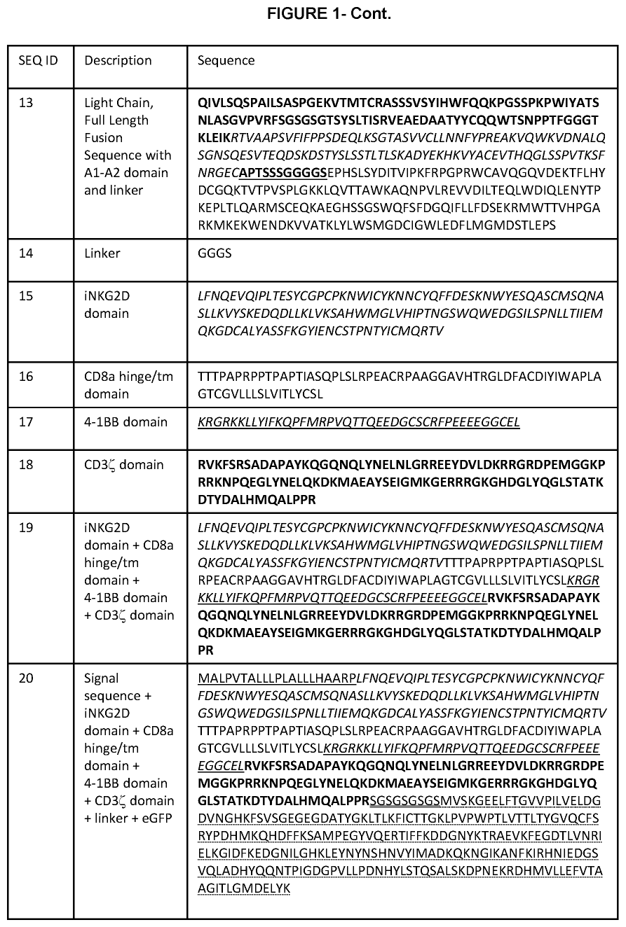

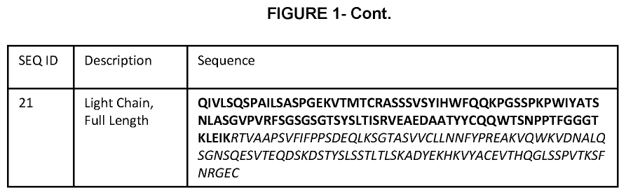

### DETAILED DESCRIPTION

The instant disclosure provides a fusion protein comprising an antibody and the A1-A2 domain of a non-natural NKG2D ligand. The non-natural NKG2D ligand selectively binds a non-natural NKG2D receptor. In various aspects of the disclosure, the fusion protein is used in connection with CAR-T cells displaying the non-natural NKG2D receptor to which the A1-A2 domain binds, thereby providing a powerful system for delivering a tailored CAR-T cell therapy which overcomes many of the disadvantages of current CAR-T cell based therapeutics. Unlike currently available CAR-T cell-based therapies, the fusion protein and system of the disclosure allows for flexible targeting to direct T cell activity to antigen of choice, multiplex capabilities to reduce the potential for antigen-loss related relapse, dose control for differential engagement of CAR-T cells, and selective delivery of modulatory agents to CAR-expressing cells.

The disclosure provides an antibody fusion protein comprising (i) heavy chains comprising variable region sequences of SEQ ID NO: 1 and (ii) light chains comprising variable region sequences of SEQ ID NO: 8. The light chains are fused at the C-terminus to an A1-A2 domain comprising the amino acid sequence of SEQ ID NO: 11. The heavy chain variable region and light chain variable region of the instant antibody fusion protein are those of Rituximab, a chimeric monoclonal antibody (IgG1 kappa immunoglobulin) that binds CD20, a surface antigen displayed on B cells. Rituximab is further described in, e.g., U.S. Pat. Nos. 5,736,137; 5,776,456; and 5,843,439. B cells play a role in the pathogenesis of certain autoimmune diseases and cancers, and Rituximab is effective in targeting and killing B cells to achieve a beneficial effect in a variety of disorders. For example, Rituximab has shown efficacy in treating cancers, such as leukemias (e.g., Hairy Cell Leukemia (HCL) and Chronic Lymphocytic Leukemia (CLL) and lymphomas (e.g., Non-Hodgkins Lymphoma (NHL, such as Diffuse Large B-cell Lymphoma (DLBCL), Burkitt Lymphoma (BL), Mantel cell Lymphoma (MCL), and follicular lymphoma). Rituximab also demonstrated efficacy in treating autoimmune disorders, such as rheumatoid arthritis, multiple sclerosis, systemic lupus erythematosus (SLE), chronic inflammatory demyelinating polyneuropathy, and autoimmune-associated anemias. Rituximab also has been approved for the treatment of Granulomatosis with Polyangiitis (GPA) (Wegener's Granulomatosis) and Microscopic Polyangiitis (MPA).

The term “antibody” as used herein refers to immunoglobulins with full length heavy chains and light chains. The antibody of the disclosure is an IgG antibody, which includes four highly conserved subclasses (IgG1, IgG2, IgG3, and IgG4), which generally differ in their constant regions (e.g., in the hinge and/or CH2 domain). Optionally, the antibody fusion protein of the disclosure comprises an IgG1 antibody, the constant region of which may be modified to reduce or inactivate the antibody's ability to trigger antibody-dependent cell cytolysis (ADCC) (e.g., by introducing D265A/D297A substitutions into the Fc domain). In various aspects, the heavy chains of the antibody fusion protein comprise constant domains comprising the amino acid sequence of SEQ ID NO: 3. The disclosure also contemplates antibody fusion proteins wherein the heavy chains comprise a constant region comprising the amino acid sequence of SEQ ID NO: 2. The disclosure also contemplates antibody fusion proteins wherein the heavy chains comprise an amino acid sequence at least 90% identical or at least 95% identical to SEQ ID NO: 3 but wherein the amino acids at positions 234, 235, and 329 within SEQ ID NO: 3 are alanine. In some aspects, the antibody fusion protein comprises heavy chains of SEQ ID NO: 7. In this regard, the disclosure provides an antibody fusion protein comprising light chains of SEQ ID NO: 21 and heavy chains of SEQ ID NO: 7. In other aspects, the antibody fusion protein comprises heavy chains of SEQ ID NO: 6 In this regard, the disclosure contemplates an antibody fusion protein comprising light chains of SEQ ID NO: 21 and heavy chains of SEQ ID NO: 6.

The light chains of the antibody comprise variable region sequences of SEQ ID NO: 8. Optionally, the light chains comprise a constant region comprising the amino acid sequence of SEQ ID NO: 9 (or a sequence at least about 90% identical or 95% identical to SEQ ID NO: 9). Thus, in various aspects, the light chains of the antibody fusion protein of the disclosure comprise SEQ ID NO: 8 and SEQ ID NO: 9 (SEQ ID NO: 21).

The light chains are fused at the C-terminus to an NKG2D ligand A1-A2 domain comprising the amino acid sequence of SEQ ID NO: 11. NKG2D is an activating receptor expressed as a type II homodimeric integral membrane protein on Natural Killer (NK) cells, some myeloid cells, and certain T cells. Human NKG2D has eight distinct natural MIC ligands (MICA, MICB, ULBP1 through ULBP6) that are upregulated on the surface of cells in response to a variety of stresses and their differential regulation provides the immune system a means of responding to a broad range of emergency cues with minimal collateral damage. Groh et al., Proc. Natl. Acad. Sci. U.S.A. 93, 12445-12450 (1996); Zwirner et al., Hum. Immunol. 60, 323-330 (1999); and Spies et al., Nat. Immunol. 9, 1013-1015 (2008). The structure of the NKG2D ectodomain, several soluble ligands, and the bound complex of ligands to the ectodomain have been solved, revealing a saddle-like groove in the homodimer interface which engages the structurally conserved A1-A2 domains of the ligands that are otherwise of disparate amino acid identity. Li et al., Nat. Immunol. 2, 443-451 (2001); Radaev et al., Immunity 15, 1039-1049 (2001); Zuo et al., Sci Signal 10, (2017); and McFarland et al., Immunity 19, 803-812 (2003). The “A1-A2 domain” of the instant disclosure is not a naturally-occurring A1-A2 domain, but comprises an amino acid sequence which binds a mutated version of an NKG2D ectodomain but does not bind wild-type NKG2D (wtNKG2D) (or at least does not bind wtNKG2D in such a manner to be biologically relevant in vivo). This orthogonal A1-A2 domain, which is based on the U2S3 domain described in the Example, is fused to the light chain of the antibodies described herein to generate a bispecific antibody fusion protein which binds both CD20 and mutated NKG2D ectodomain. This format (antibodies fused to an A1-A2 domain) is also referred to herein as a “MicAbody.” As explained in more detail below, fusion of the A1-A2 domain to the C terminus of the light chain amino acid sequence resulted in superior activity compared to fusion of the A1-A2 domain on the heavy chains of the antibody of the disclosure. The superior properties of the placement of the domain on the antibody fusion protein described herein could not have been predicted prior to the study described in the Example.

In various aspects, the A1-A2 domain is fused to the C-terminus of the light chain via a linker, optionally a linker comprising (or consisting of) SEQ ID NO: 10. As explained in more detail below, the linker of SEQ ID NO: 10 produced a MicAbody which unexpectedly outperformed other antibody fusion constructs in terms of B cell cytotoxicity. Thus, in exemplary aspects of the disclosure, the antibody fusion protein of the disclosure comprises light chains comprising a variable region sequence of SEQ ID NO: 8 and the A1-A2 domain of SEQ ID NO: 11 fused to the C-terminus of the light chain via a linker sequence of SEQ ID NO: 10, optionally comprising the light chain constant region of SEQ ID NO: 9. In this regard, in various aspects of the disclosure, the light chains of the antibody fusion protein comprise the amino acid sequence of SEQ ID NO: 13.

The disclosure provides an antibody fusion protein comprising light chains of SEQ ID NO: 13 and heavy chains of SEQ ID NO: 7. The disclosure also provides an antibody fusion protein comprising light chains of SEQ ID NO: 13 and heavy chains of SEQ ID NO: 6. Methods of making antibodies and antibody fusion proteins are known in the art and described, e.g., in the Example below.

The disclosure also provides a kit comprising one or more containers comprising the antibody fusion protein described herein. The kit may further comprise instructions and written information on indications and usage of the antibody fusion protein. Syringes, e.g., single use or pre-filled syringes, sterile sealed containers, e.g. vials, bottle, vessel, and/or kits or packages comprising the antibody fusion protein, optionally with suitable instructions for use, are also contemplated. In a further aspect, the disclosure provides an article of manufacture, or unit dose form, comprising: (a) a composition of matter comprising the antibody fusion protein described herein; (b) a container containing said composition; and (c) a label affixed to said container, or a package insert included in said container referring to the use of said antibody fusion protein in the treatment of a disease or disorder (e.g., cancer). Also provided herein are compositions comprising the antibody fusion protein (and, in various aspects, mammalian cells expressing a CAR as described herein) and a pharmaceutically acceptable carrier, excipient or diluent. In exemplary aspects, the composition is a sterile composition.

The disclosure further provides a system or kit comprising components of a cell therapy regimen targeting CD20-displaying cells. In various aspects, the first component is the antibody fusion protein described herein, i.e., a bispecific, antibody-based fusion protein that binds both CD20 and a CAR comprising an NKG2D ectodomain. The second component is a mammalian cell (e.g., human cell) that is genetically modified to express a chimeric antigen receptor (CAR) that is itself inert (i.e., unarmed CAR-T). In various aspects, the mammalian cell is a lymphocyte or a macrophage, e.g., a human lymphocyte (such as human T cell) or a human macrophage. In various aspects, the second component is a human NK (natural killer) cell (e.g., an autologous human NK cell); disclosure herein with reference to T cells also applies to NK cells. The kit comprises one or more containers comprising mammalian cells expressing the CAR and one or more containers comprising the antibody fusion protein. A kit may further comprise instructions and written information on indications and usage of the components described herein.

“Chimeric antigen receptor” or “CAR” refers to an artificial immune cell receptor that is engineered to recognize and bind to an antigen expressed by a target cell, such as a tumor cell. Generally, a CAR is designed for a T cell and is a chimera of a signaling domain of the T cell receptor (TCR) complex and an antigen-recognizing domain (e.g., a single chain fragment (scFv) of an antibody or other antibody fragment). See, e.g., Enblad et al., Human Gene Therapy. 2015; 26(8):498-505. T cells and NK-cells can be modified using gene transfer techniques to directly and stably express on their surface transmembrane signaling receptors that confer novel antigen specificities. See, e.g., Gill & June, Immunological Reviews 2015. Vol. 263: 68-89; Glienke et al., Front. Pharmacol. doi: 10.3389/fphar.2015.00021. There are various formats of CARs, each of which contains different components. “First generation” CARs join an antigen binding domain to the CD3zeta intracellular signaling domain of the T-cell receptor through hinge and transmembrane domains. “Second generation” CARs incorporate an additional domain, e.g., CD28, 4-1BB (41BB), or ICOS, to supply a costimulatory signal. “Third generation” CARs contain two costimulatory domains fused with the TCR CD3zeta chain. Third generation costimulatory domains may include, e.g., a combination of CD3zeta, CD27, CD28, 4-1BB, ICOS, or OX40. CARs so constructed can trigger, e.g., T cell activation upon binding the targeted antigen in a manner similar to an endogenous T cell receptor, but independent of the major histocompatibility complex (MHC).

The chimeric antigen receptor of the disclosure comprises, as the “antigen binding domain” of the CAR, a mutated NKG2D ectodomain that is incapable of engaging natural ligands. Mutation of the NKG2D ectodomain is further described in, e.g., Culpepper et al., Mol. Immunol. 48, 516-523 (2011) and the Example. The mutated NKG2D is referred to herein as “INKG2D.” In various aspects, the iNKG2D domain comprises the amino acid sequence of SEQ ID NO: 15. The ectodomain is preferably associated with a transmembrane domain, an intracellular domain of a costimulatory molecule (e.g., 4-1BB or CD28), and/or a T cell receptor intracellular signaling domain. For example, in exemplary aspects of the disclosure, the iNKG2D ectodomain is fused to a CD8a hinge/transmembrane domain (e.g., comprising or consisting of the sequence of SEQ ID NO: 16), a 4-1BB domain (e.g., comprising or consisting of the sequence of SEQ ID NO: 17), and/or a CD3ζ domain (e.g., comprising or consisting of the sequence of SEQ ID NO: 18). In various aspects, the CAR comprises all of these components (e.g., SEQ ID NOs: 15-18 or SEQ ID NO: 19).

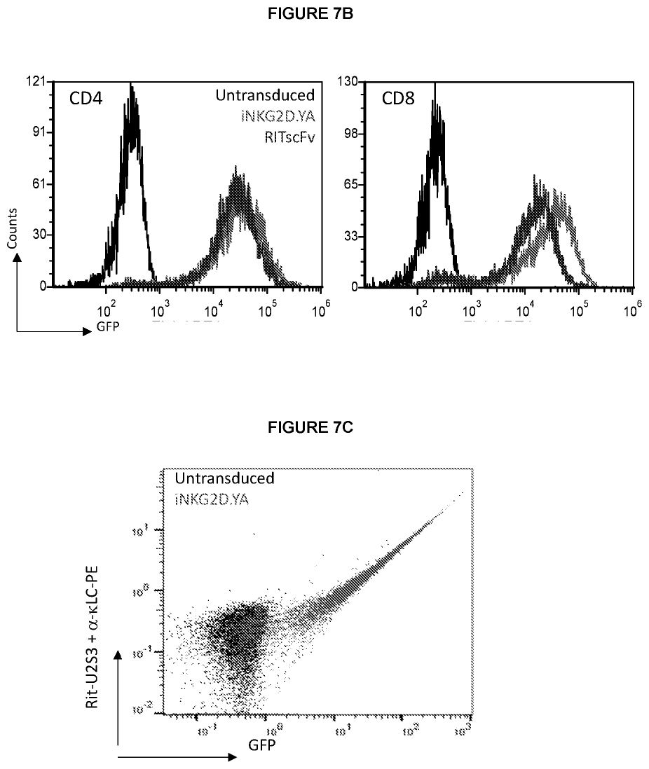

Because the CAR is inert, the CAR can only form a productive immunologic synapse with a target cell displaying the antigen and activate cytolysis when it is “armed” with its cognate antibody fusion protein noncovalently bound to its receptor. The CAR-expressing cell is referred to herein as “convertibleCAR.” An example of the system is illustrated in FIG. 7A. The antibody fusion protein described herein is capable of activating iNKG2D-CAR-expressing cells (e.g., T cells) only in the presence of cells expressing CD20. When used with additional MicAbodies that target other antigens (i.e., antibody fusion proteins having different variable regions that bind different cell surface antigens), convertibleCAR-T cells can be targeted to different antigens simultaneously or sequentially to mediate cytolysis; this approach can help address, e.g., tumor resistance and escape as a result of target antigen loss without having to create, expand and infuse multiple different autologous CAR cells. This highly modular convertibleCAR system expands the potential of adoptive cell therapies and overcomes many of disadvantages of existing cell therapies, including severe systemic toxicity, antigen escape, and limited and uncontrolled persistence of current CAR-T and CAR-NK cell therapeutics. Additionally, since a single CAR may be used in a variety of contexts (because the targeting specificity is determined by the antibody fusion protein administered, not the CAR), cell manufacturing is simplified and less expensive.

A CAR cellular therapy may be an immunotherapy utilizing a subject or a patient's own immune cells that are engineered to be able to produce a particular CAR(s) on their surface. In some situations, cells (e.g., T cells) are collected from the body of a subject or a patient via apheresis. The cells (e.g., T cells) collected from the body are then genetically engineered to produce a particular chimeric antigen receptor on their surface. The CAR-expressing cells are expanded by growth in a laboratory and then administered to the subject or patient, or another subject or patient. The CAR-expressing cells will recognize and kill cells (e.g., cancer cells) that express the targeted antigen on their surface. The cells may be isolated from the subject which will be recipient of the therapy, or may be isolated from a donor subject that is not ultimate recipient of the therapy. In various aspects, the cells are autologous CD4+ and CD8+ T cells.

The disclosure further provides a method of treating a subject for a disease or disorder associated with cells expressing CD20, such as cancer (CD20-positive cancers). The method comprises administering to the subject the CAR-expressing cell described herein (e.g., a T cell or NK cell expressing the iNKG2D-based CAR described herein) and administering to the subject the antibody fusion protein described herein. Examples of cancers include, but are not limited to, leukemias and lymphomas, such as Hairy Cell Leukemia, Chronic Lymphocytic Leukemia, and Non-Hodgkins Lymphoma (e.g., Diffuse Large B-cell Lymphoma, Burkitt Lymphoma, Mantel cell Lymphoma, and follicular lymphoma).

As used herein, the term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease or disorder can provide any amount or any level of treatment. Furthermore, the treatment provided by the method may include treatment of one or more conditions or symptoms or signs of the disease being treated. For instance, the treatment method of the present disclosure may inhibit one or more symptoms of the disease. Also, the treatment provided by the methods of the present disclosure may encompass slowing the progression of the disease.

Treatment for cancer may be determined by any of a number of ways. Any improvement in the subject's wellbeing is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about an 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease, (8) decrease in levels of cancer markers; (9) an increased patient survival rate; and/or (10) some relief from one or more symptoms associated with the disease or condition (e.g., pain). In addition, treatment efficacy also can be characterized in terms of responsiveness to other immunotherapy treatment or chemotherapy. In various aspects, the methods of the disclosure further comprise monitoring treatment in the subject.

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammal is of the order Primate, Ceboid, or Simoid (monkey) or of the order Anthropoid (humans and apes). In some aspects, the mammal is a human. Therapeutic compositions may be delivered to a subject using any of a variety of routes, including parenteral, topical, oral, intrathecal or local administration. Indeed, a composition may be administered subcutaneously, intracutaneously, intradermally, intravenously, intraarterially, intratumorally, parenterally, intraperitoneally, intramuscularly, intraocularly, intraosteally, epidurally, intradurally, intratumorally and the like.

The disclosure also provides (i) nucleic acid molecules (i.e., isolated nucleic acids) encoding the light chain of the antibody fusion protein described herein and (ii) nucleic acid molecules (i.e., isolated nucleic acids) encoding the heavy chain of the antibody fusion protein described herein, as well as compositions comprising (i) and/or (ii). Nucleic acids of the disclosure include nucleic acids encoding any of the amino acid sequences disclosed herein, as well as nucleic acids comprising nucleotide sequences having at least 80%, more preferably at least about 90%, more preferably at least about 95%, and most preferably at least about 98% identity to nucleic acids of the disclosure (i.e., the nucleic acid sequences set forth in the sequence listing). Nucleic acids of the disclosure also include complementary nucleic acids. In some instances, the sequences will be fully complementary (no mismatches) when aligned. In other instances, there may be up to about a 20% mismatch in the sequences. The disclosure provides nucleic acid molecules comprising nucleic acid sequences encoding both a heavy chain and a light chain of an antibody fusion protein of the disclosure.

Nucleic acids of the disclosure can be cloned into an expression vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the disclosure may further comprise regulatory sequences, for example, an internal ribosomal entry site. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell. The expression vector can be introduced into a cell by transfection, for example.

Recombinant host cells comprising the nucleic acid molecules (optionally contained in expression vectors) also are provided. The recombinant host cell may be a prokaryotic cell, for example an E. coli cell, or a eukaryotic cell, for example a mammalian cell or a yeast cell. Yeast cells include, e.g., Saccharomyces cerevisiae, Schizosaccharomyces pombe, and Pichia pastoris cells. Mammalian cells include, for example, VERO, HeLa, Chinese hamster Ovary (CHO), W138, baby hamster kidney (BHK), COS-7, MDCK, human embryonic kidney line 293, African green monkey kidney cells, and COS cells. Recombinant protein-producing cells of the disclosure also include any insect expression cell line known, such as for example, Spodoptera frugiperda cells. In one embodiment, the cells are mammalian cells, such as CHO cells.

A method of producing an antibody fusion protein further is provided by the disclosure. The method comprises culturing a host cell (an isolated host cell) comprising a nucleic acid molecule comprising a nucleotide sequence encoding the light chain of the antibody fusion protein and a nucleic acid molecule comprising a nucleotide sequence encoding the heavy chain of the antibody fusion protein. The method further comprises recovering the antibody fusion protein. Culture conditions and methods for generating antibody proteins are known in the art. Similarly, protein purification methods are known in the art and utilized herein for recovery of recombinant proteins from cell culture media. In some aspects, methods for protein and antibody purification include filtration, affinity column chromatography, cation exchange chromatography, anion exchange chromatography, and concentration. Optionally, the method comprises formulating the antibody fusion protein.

The following example is given merely to illustrate the present invention and not in any way to limit its scope.

### EXAMPLE

This Example describes exemplary methods of producing an antibody fusion protein of the disclosure and NKG2D ectodomain-comprising CAR-T cells. The Example further demonstrates the ability of an antibody fusion protein comprising the variable region sequences from Rituximab and comprising an A1-A2 domain fused to the C-terminus of the light chain to selectively bind a CAR-T cell comprising the amino acid sequences of SEQ ID NOs: 15-18, and the ability of the antibody fusion protein and CAR-T cell combination to kill CD20-bearing cancer cells in vivo.

### Materials and Methods

Cloning, expression, and purification: The wild-type ectodomain of NKG2D (UniProtKB P26718, residues 78-216; https://www.uniprot.org) was expressed as a fusion to the C-terminus of human IgG1 Fc via a short factor Xa recognizable Ile-Glu-Gly-Arg linker (Fc-wtNKG2D). Inert NKG2D variants comprising either a single Y152A (iNKG2D.YA) or double Y152A/Y199A substitution (INKG2D.AF) were generated by PCR-mediated mutagenesis or synthesized (gBlocks®, IDT). DNA constructs for Fc-NKG2D molecules were expressed in Expi293™ cells (Thermo Fisher Scientific) and dimeric secreted protein was purified by Protein A affinity chromatography (Pierce™ #20334, Thermo Fisher). Eluted material was characterized and further purified by size-exclusion chromatography (SEC) on an ÄKTA Pure system using Superdex 200 columns (GE Life Sciences). Correctly assembled, size-appropriate monomeric material was fractionated into phosphate-buffered saline (PBS).

The A1-A2 domains of human MICA*001 (UniProtKB Q29983, residues 24-205), MICB (UniProtKB Q29980.1, 24-205), ULBP1 (UniProtKB Q9BZM6, 29-212), ULBP2 (UniProtKB Q9BZM5, 29-212), ULBP3 (UniProtKB Q9BZM4, 30-212), ULBP5 (NCBI accession NP_001001788.2, 29-212), and ULBP6 (UniProtKB, 29-212) were cloned with a C-terminal 6×-His tag. Monomeric protein was purified from Expi293™ supernatants Ni-NTA resin (HisPur™, Thermo Fisher) and eluted material exchanged into PBS with Sephadex G-25 in PD-10 Desalting Columns (GE Life Sciences).

MIC ligands and orthogonal variants were cloned by ligation-independent assembly (HiFi DNA Assembly Master Mix, NEB #E2621) as fusions to the C-terminus of either the kappa light-chain or the heavy-chain of human IgG1 antibodies via either an APTSSSGGGGS or GGGS linker, respectively. Additionally, D265A/N297A (Kabat numbering) mutations were introduced into the CH2 domain of the heavy chain of all antibody and MicAbody clones to eliminate antibody-dependent cell cytotoxicity (ADCC) function. Heavy-and light-chain plasmid DNAs (in the mammalian expression vector pD2610-V12 (ATUM) for a given antibody clone were co-transfected into Expi293™ cells and purified by Protein A. For any monoclonal antibody fusion generated, the appropriate VL or VH domains were swapped into either the kappa light-chain or an ADCC-deficient IgG1 heavy-chain.

Inert NKG2D and orthogonal ligand engineering: Bio-layer interferometry (BLI) with the ForteBio Octet system (Pall ForteBio LLC) was implemented to validate loss of wild-type MIC ligand binding by iNKG2D. Fc-wtNKG2D, Fc-INKG2D.YA, or Fc-INKG2D.AF was captured on anti-human IgG Fc capture (AHC) biosensor tips and association/dissociation kinetics monitored in a titration series of monomeric MIC-His ligands. Additionally, ELISA (enzyme-linked immunosorbent assay) binding assays were performed with MICA-Fc, MICB-Fc, ULBP1-Fc, ULBP2-Fc, ULBP3-Fc, or ULBP4-Fc (R&D Systems) coated onto microtiter plates, a titration of biotinylated Fc-wtNKG2D or Fc-iNKG2D.YA, detected with streptavidin-HRP (R&D Systems #DY998), and developed with 1-Step Ultra TMB ELISA (Thermo Fisher #34208).

Phage display was employed to identify orthogonal ULBP2 A1-A2 variants that exhibited exclusive binding to either INKG2D.YA or INKG2D.AF. Synthetic NNK (where N=A/C/G/T and K=G/T, resulting in representation of all 20 amino acids without stop codons) DNA libraries were generated targeting the codons of helix 2 (residues 74-78, numbering based upon mature protein) or helix 4 (residues 156-160) that in the bound state are positioned in close proximity to the Y152 positions on the natural NKG2D receptor45. Müller et al., PLOS Pathog. 6, e1000723 (2010). Libraries exploring helix 2 alone, helix 4 alone, or the combination were cloned as fusions to the pIII minor coat protein of M13 phage, and phage particles displaying the mutagenized A1-A2 domain variants were produced in SS320 E. coli cells according to standard methods. These A1-A2 phage libraries were captured with either biotinylated Fc-INKG2D.YA or Fc-iNKG2D.AF protein (EZ-Link™ NHS-Biotin Kit, Thermo Fisher #20217) and enriched by cycling through four rounds of selection with increasing concentrations of non-biotinylated Fc-wtNKG2D competitor. Positive phage clones were verified for preferential binding to plate-bound Fc-iNKG2D.YA or Fc-iNKG2D.AF versus Fc-wtNKG2D by spot ELISA and bound phage detected with biotinylated M13 phage coat protein monoclonal antibody E1 (Thermo Fisher #MA1-34468) followed by incubation with streptavidin-HRP.

Phage variants were sequenced then cloned as human IgG1 monoclonal antibody fusions for additional validation. To confirm that selectivity of orthogonal variants was maintained in the bivalent MicAbody format, ELISA wells were coated with 1 ug/mL Fc-wtNKG2D, Fc-iNKG2D. YA, or Fc-iNKG2D.AF, and bound MicAbody was detected with an HRP-conjugated mouse-anti-human kappa chain antibody (Abcam #ab79115). Affinity of both monomeric and antibody-fused ULBP2 variants was also determined by Octet analysis as described above.

Generation of convertibleCAR-T cells: Human-codon optimized DNA (GeneArt, Thermo Fisher) comprising the CD8α-chain signal sequence, NKG2D variant, CD8α hinge and transmembrane domains, 4-1BB, CD3ζ, and eGFP were cloned into the pHR-PGK transfer plasmid for second generation Pantropic VSV-G pseudotyped lentivirus production along with packaging plasmids pCMVdR8.91 and pMD2.G48. The VH and VL domains of Rituximab separated by a (GGGGS)₃ linker were substituted for the NKG2D module to generate the rituximab scFv-based CAR (RITscFv-CAR). For each batch of lentivirus produced, 6×10⁶ Lenti-X 293T (Takara Bio #632180) cells were seeded in a 10 cm dish the day prior to transfection. Then 12.9 μg pCMVdR8.91, 2.5 μg pMD2.G and 7.2 μg of the pHR-PGK-CAR constructs were combined in 720 μl Opti-MEM™ (Thermo Fisher #31985062), then mixed with 67.5 μl of Fugene HD (Promega Corp. E2311), briefly vortexed, and incubated at room temperature for 10 minutes before adding to the dish of cells. After two days, supernatants were collected by centrifugation and passed through 0.22 μm filters. 5× concentrated PEG-6000 and NaCl were added to achieve final concentrations of 8.5% PEG-6000 (Hampton Research #HR2-533) and 0.3 M NaCl, incubated on ice for two hours, then centrifuged at 4° C. for 20 minutes. Concentrated viral particles were resuspended in 0.01 volume of PBS, and stored frozen at −80° C.

For primary human T cell isolation, a Human Peripheral Blood Leuko Pak (Stemcell Technologies #70500.1) from an anonymous donor was diluted with an equivalent volume of PBS+2% FBS, then centrifuged at 500×g for 10 minutes at room temperature. Cells were resuspended at 5×10⁷ cells/ml in PBS+2% FBS and CD4+ or CD8+ cells enriched by negative selection (Stemcell EasySep™ Human CD4 T Cell Isolation Kit #17952 or EasySep Human CD8 T Cell Isolation Kit #17953) by addition of 50 μl of isolation cocktail per ml of cells and incubating for five minutes at room temperature. Subsequently, 50 μl of RapidSpheres™ were added per ml of cells and samples topped off (to each 21 mL cells, 14 mL of PBS). Cells were isolated for 10 minutes with an EasySEP™ magnet followed by removal of buffer while maintaining the magnetic field. Enriched cells were transferred into new tubes with fresh buffer and the magnet reapplied for a second round of enrichment after which cells were resuspended, counted, and cryopreserved at 10-15×10⁶ cells/cryovial (RPMI-1640, Corning #15-040-CV; 20% human AB serum, Valley Biomedical #HP1022; 10% DMSO, Alfa Aesar #42780).

To generate CAR-T cells, one vial of cryopreserved cells was thawed and added to 10 ml T cell medium “TCM” (TexMACS medium, Miltenyi 130-097-196; 5% human AB serum, Valley Biomedical #HP1022; 10 mM neutralized N-acetyl-L-Cysteine; 1× 2-mercaptoethanol, Thermo Fisher #21985023, 1000×; 45 IUe/ml human IL-2 IS “rhIL-2”, Miltenyi #130-097-746) added at time of addition to cells. Cells were centrifuged at 400× g for 5 minutes then resuspended in 10 ml TCM and adjusted to 1×10⁶/ml and plated at 1 ml/well in a 24 well plate. After an overnight rest 20 μL of Dynabeads™ Human T-Activator CD3/CD28 (Thermo Fisher #1131D) were added per well and incubated for 24 hours. Concentrated lentiviral particles (50 μL) were added per well, cells incubated overnight, then transferred to T25 flasks with an added 6 ml TCM. After three days of expansion, Dynabeads were removed (MagCellect magnet, R&D Systems MAG997), transduction efficiency assessed by flow cytometry for GFP, back-diluted to 5×10⁵ cells/mL, and cell density monitored daily to ensure they did not exceed 4×10⁶ cells/ml. When necessary, surface expression of iNKG2D was correlated with GFP expression using a MicAbody and detecting with PE-anti-human kappa chain (Abcam #ab79113) or by directly conjugating the Rituximab-MicAbody to Alexa Fluor 647 (Alexa Fluor Protein Labeling Kit #A20173, Thermo Fisher). The amount of iNKG2D expression on the surface of convertible CAR-CD8 cells was quantified using Alexa Fluor 647 conjugated Rituximab-MicAbody, and median fluorescence intensity was correlated with Quantum™ MESF 647 beads (Bangs Laboratories #647). All flow cytometry was performed on either Bio-Rad S3e Cell Sorter or Miltenyi MACSQuant Analyzer 10 instruments.

Cell lines and in vitro assays: Ramos human B cell lymphoma cells (ATCC #CRL-1596) were cultured in RPMI supplemented with 20 mM HEPES and 10% FBS. The mouse colon carcinoma line CT26 transfected to express human Her2 were also used. No additional mycoplasma testing nor authentication was performed except to verify by flow cytometry that target antigens were expressed.

For calcein-release assays, tumor cells were centrifuged and resuspend in 4 mM probenecid (MP Biomedicals #156370)+25 UM calcein-AM (Thermo Fisher #C1430) in T cell medium at 1-2×10⁶ cells/ml for one hour at 37° C., washed once, and adjusted to 8×10⁵ cells/ml. CD8+ CAR-T cells were pelleted and resuspended in 4 mM probenecid with 60 IUe/ml IL-2 in TCM at 4×10⁶ cells/mL then adjusted according to the desired effector:target ratio (unadjusted for transduction efficiency). 25 UL target cells were plated followed by 25 UL medium or diluted MicAbody. Then 100 μL medium (minimum lysis), medium +3% Triton-X 100 (maximum lysis), or CAR-T cells were added and plates incubated at 37° C. for two hours. Cells were pelleted and 75 μL supernatant transferred to black clear-bottom plates and fluorescence (excitation 485 nm, emission cutoff 495 nm, emission 530 nm, 6 flashes per read) acquired on a Spectramax M2e plate reader (Molecular Devices). For experiments with armed convertibleCAR-CD8+s, T cells were pre-incubated at 37° C., with either saturating (5 nM) or a titration of MicAbody for 30 minutes before washing to remove unbound MicAbody and co-culturing with calcein-loaded target cells.

In order to quantify the target-dependent activation of T-cells, experiments were set up as described above except that calcein-preloading was omitted and assays set up in T cell medium without IL-2 supplementation. After 24 hours co-culture, supernatants were harvested and stored at −80° C. until the amount of liberated cytokine could be quantified by ELISA MAX™ Human IL-2 or Human IFN-g detection kits (BioLegend #431801 and #430101).

The MicAbody binding curve data were generated by ProMab Biotechnologies, Inc. (Richmond, CA). 3×10⁵ convertibleCAR-CD8+ cells were plated in 96-wells V-bottom plates and incubated with labeled Alexa Fluor 647 labeled Rituximab. LC-U2S3 MicAbody for 30 minutes at room temperature in a final volume of 100 μL RPMI+1% FBS with a titration curve starting at 200 nM. Cells were then rinsed and median fluorescence intensity determined for each titration point by flow cytometry.

Animal studies: For PK analysis of serum levels of MicAbodies, six-week old female NSG mice (NOD.Cg-Prkdcscid IL2rgtm1Wjl/SzJ, The Jackson Laboratory #005557) were injected intravenously (IV) with 100 μg of either parent rituximab antibody (ADCC-defective), heavy-chain U2S3 fusion of rituximab (Rituximab.HC-U2S3), or light-chain fusion (Rituximab.LC-U2S3). Collected sera were subjected to ELISA by capturing with human anti-rituximab idiotype antibody (HCA186, Bio-Rad Laboratories), detected with rat-anti-rituximab-HRP antibody (MCA2260P, Bio-Rad), and serum levels interpolated using either a rituximab or Ritxumab-U2S3 standard curve. PK analysis of U2S3-hFc-mutIL2 was performed in NSG mice by IP injection of 60 μg followed by regular serum collection. Samples were examined by ELISA capturing with Fc-iNKG2D and detecting with biotinylated rabbit-anti-human IL-2 polyclonal antibody (Peprotech #500-P22BT) followed by incubation with streptavidin-HRP. Half-lives were calculated in GraphPad Prism based upon the β-phase of the curve using a nonlinear regression analysis, exponential, one-phase decay analysis with the plateau constrained to zero.

For disseminated Raji B cell lymphoma studies, six-week old female NSG mice were implanted IV with Raji cells (ATCC #CCL-86) stably transfected to constitutively express luciferase from Luciola italica (Perkin Elmer RediFect Red-FLuc-GFP #CLS960003). Initiation of treatment administration is detailed in each in vivo study figure. For all experiments, CD4 and CD8 primary human T cells were independently transduced, combined post-expansion at a 1:1 mixture of CD4:CD8 cells without normalizing for transfection efficiency between cell types or CAR constructs, and the mixture validated by flow cytometry prior to IV injection. Administration of MicAbody or control antibody was by the intraperitoneal (IP) route unless otherwise specified, and in vivo imaging for bioluminescence was performed with a Xenogen IVIS system (Perkin Elmer). Animals were bled regularly to monitor human T cell dynamics by flow cytometry, staining with APC Anti-Human CD3 (clone OKT3, #20-0037-T100, Tonbo Biosciences), monitoring GFP, and examining cell-associated MicAbody levels with biotinylated Anti-Human F(ab′)2 (#109-066-097, Jackson ImmunoResearch Laboratories Inc.) followed by Streptavidin-PE detection (BD #554061). Serum ELISAs to monitor MicAbody levels was performed as described above.

For subcutaneous tumor studies 1×10⁶ Raji cells were implanted in matrigel on the right flank of six-week old female NSG mice and therapy initiated when tumors reached 70-100 mm³. For the cohort that received armed convertibleCAR-T cells, the cells were incubated with 5 nM Rituximab.LC-U2S3 MicAbody ex vivo for 30 minutes at room temperature before washing and final mixing to achieve the desired 1:1 CD4:CD8 ratio and cell concentration. Arming was confirmed by flow cytometry with the biotinylated Anti-Human F(ab′)₂ antibody and revealed a strong correlation between GFP and F(ab′)₂ MFIs. These mice did not receive a separate MicAbody administration. Caliper measurements were regularly taken to estimate tumor volume (L×W×W×0.5=mm³), and terminal tumor masses were weighed.

Complement-mediated ablation of INKG2D.AF-CAR cells: To generate Fc reagents with enhanced complement binding and targeted delivery to the T cells expressing iNKG2D.AF, the orthogonal ligand was cloned as a fusion to either the N-(U2R-Fc) or C-terminus (Fc-U2R) of human IgG1 Fc via a GGGS linker with the Fc including the hinge, CH2, and CH3 domains. In addition to the wild-type Fc, the K326A/E333A21 (Kabat numbering, “AA”) and S267E/H268F/S324T/G236A/1332E20 (“EFTAE”) C1q-enhanced binding mutation sets were explored. All were expressed in Expi293T cells, purified, and fractionated as described above. Confirmatory ELISAs were performed by capturing with Fc-NKG2D.AF followed by binding U2R/Fc-variant fusions at 1 μg/mL concentration, titrating in human-C1q protein (Abcam #ab96363), then detecting with polyclonal sheep-anti-C1q-HRP antibody (Abcam #ab46191). Complement-dependent cytotoxicity (CDC) assays were performed by iQ Biosciences (Berkeley, CA). Briefly, 5×10⁴ CD8+ cells from an NKG2D.AF-CAR transduction were plated in 96-well plates and incubated for three hours with a serial dilution of each U2R/Fc-variant fusion, in triplicate, in the presence of normal human serum complement (Quidel Corporation) at a final concentration of 10% (v/v). Cells were then harvested and resuspended with SYTOX™ Red dead cell stain (Thermo Fisher) at a final concentration of 5 μg/mL and analyzed by flow cytometry. EC50 values for cytotoxicity were calculated in GraphPad prism fitted to a non-linear regression curve.

Delivery of mutant-IL2 to T cells expressing INKG2D-CAR: To generate a reagent that was monomeric for the U2S3 ligand, monomeric for a mutant IL-2 with the inability to bind IL-2Rx (mutIL2, R38A/F42K) (Heaton et al., Cancer Res. 53, 2597-2602 (1993); Sauve et al., Proc. Natl. Acad. Sci. U.S.A. 88, 4636-4640 (1991) yet retained serum stability, a heterodimeric Fc strategy was employed. Gunasekaran et al., J. Biol. Chem. 285, 19637-19646 (2010). U2S3 was fused to the N-terminus of the Fc-hinge of one chain with K392D/K409D (Kabat numbering) mutations while the mutIL2 was fused to the C-terminus of the second Fc-chain which harbored E356K/D399K mutations. Additionally, D265A/N297A mutations were introduced in both Fc chains to render the Fc ADCC-deficient. Expression in Expi293T cells and purification was as described above. Appropriately assembled U2S3-hFc-mutIL2 material was fractionated by SEC and the presence of individual size-appropriate polypeptides was confirmed by denaturing SDS-PAGE. A direct fusion between orthogonal ligand and mutIL2 expressed as a single polypeptide with a linker comprising glycine-serine linkages, a FLAG tag, and a 6×His tag was also generated and purified by Ni-NTA exchange chromatography. Ghasemi et al., Nat Commun 7, 12878 (2016). Determination of IUe activity equivalents was based on the calculation that a 4.4 M solution of wild-type IL-2 has the equivalent of 1000 IU/μL. IL-15 with a V49D mutation, which reduced binding to IL-15Ra but retained bioactivity26, was similarly formatted with U2S3. Bernard et al., J. Biol. Chem. 279, 24313-24322 (2004).

CAR-T cell proliferation in response to various cytokines or U2S3-cytokine fusions was quantified with the WST-1 Cell Proliferation Reagent (Millipore Sigma #5015944001). Briefly, CAR-T cells were pelleted and resuspended in T cell media without IL-2, dispensed into 96-well plates at 4×10⁴ cells/well, and the appropriate amount of diluted U2S3-cytokine fusions was added to achieve 30 IUe/mL or higher concentration as needed in a final assay volume of 100 μL per well. Recombinant-human IL2 and IL 15 (Peprotech #200-02 and #200-15) were included as controls. After incubation for three days at 37° C., 10 μL of WST-1 was added to each well and allowed to incubate for 30-60 minutes before quantifying intensity of color development on a plate reader. Changes in the proportion of GFP+ CAR-expressing cells in response to U2S3-cytokine fusion were monitored by flow cytometry. To monitor activation of STAT3 or STAT5 upon cytokine-fusion engagement cells were rested overnight in TCM media without IL-2 supplementation then treated with 150 IUe/mL IL-2, IL-15, U2S3-hFc-mutIL2, or U2S3-hFc-mutIL 15 for two hours before fixing and staining for intracellular phospo-STAT3 (Biolegend PE anti-STAT3 Tyr705 clone 13A3-1) and -STAT5 (BD Alexa Fluor 647 anti-STAT5 pY694 clone 47). To monitor a temporal response, treated convertibleCAR-CD8 T cells were fixed at 0, 30, 60, and 120 minutes after exposure to cytokines or U2S3-hFc-cytokine fusions then stained.

Human PBMC stimulation and immune-phenotyping studies were performed. Briefly, normal PBMCs from three donors were seeded in 96-well plates at 1×10⁵ cells/well and exposed to a 10-fold dilution series of either U2S3-hFc-mutIL2 or U2S3-hFc-wtIL2 (wild-type IL2) for four days at 37° C., with 5% CO₂. Positive controls included wells coated with anti-human CD3 (OKT2) at 2 μg/mL and rhIL-2 at 300 IUe/mL. After incubation, cells were treated with TruStain FcX block (BioLegend #422301) followed by staining with BioLegend antibody panels for proliferating T cells (CD8 clone RPA-T8 #301050, CD4 clone OKT4 #317410, CD3 clone OKT3 #300430, KI-67 #350514), and Treg cells (Fox3 clone 206D #320106, CD4 clone OKT4, CD3 clone OKT3, KI-67).

### Results

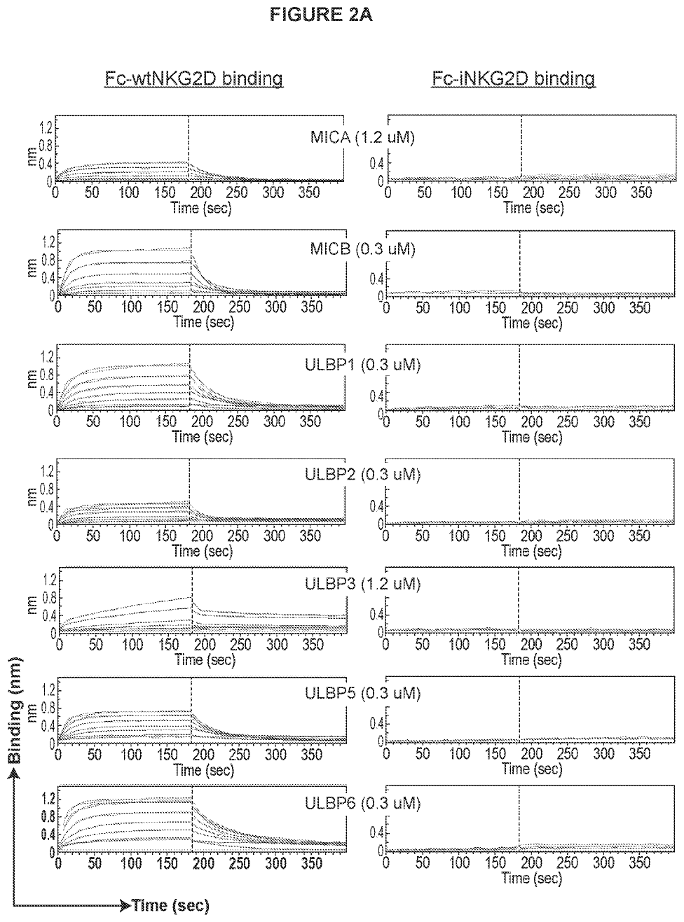

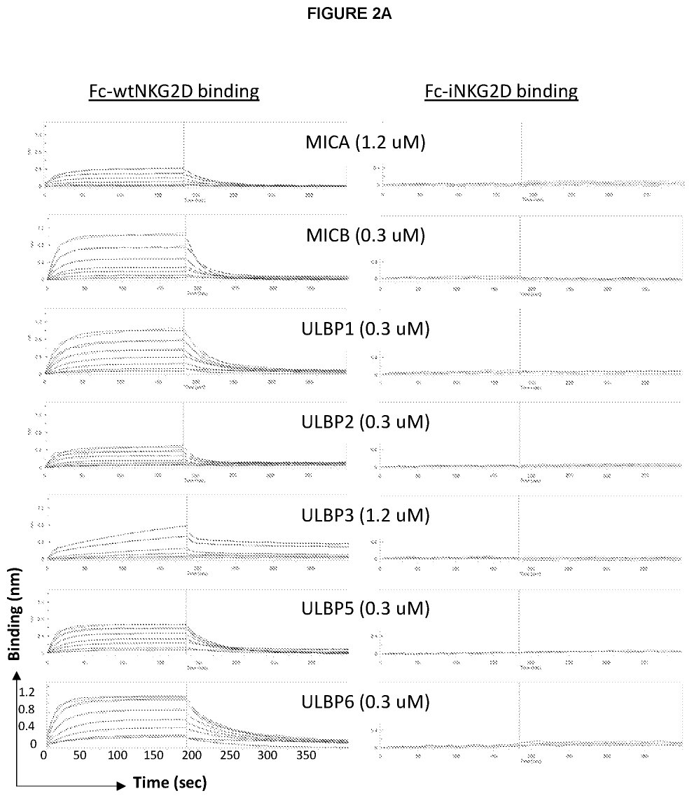

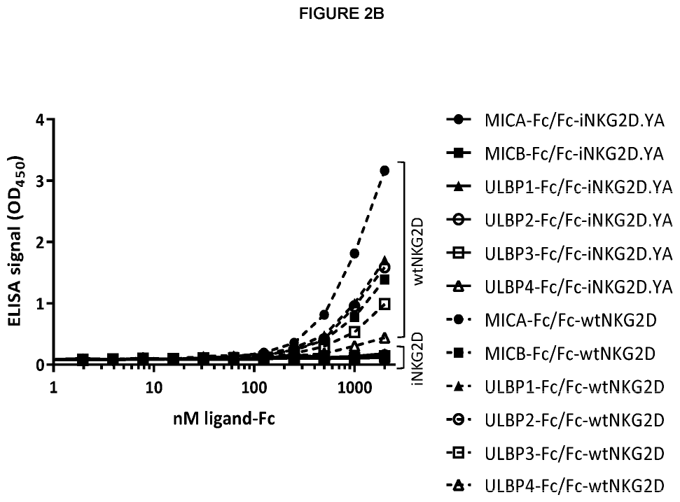

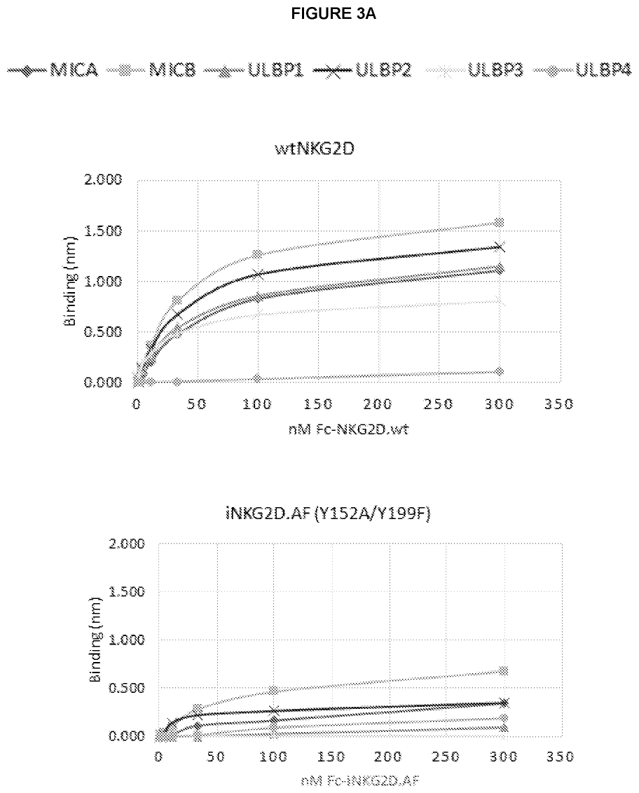

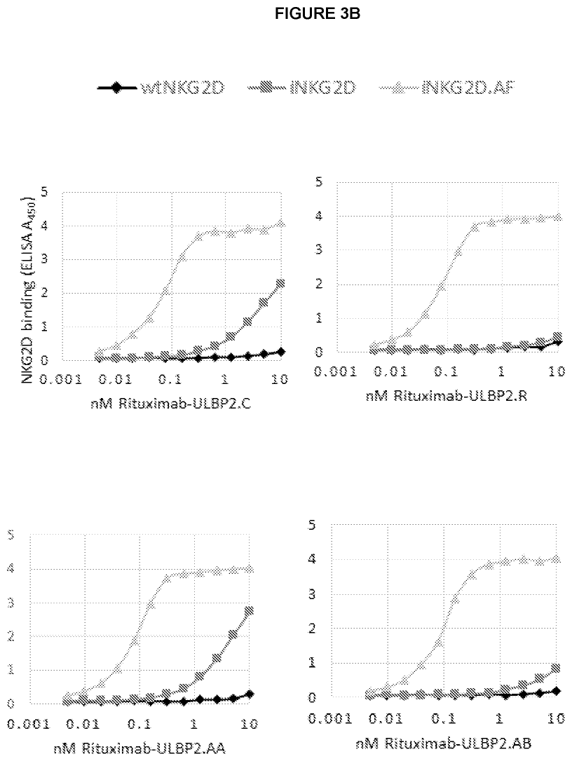

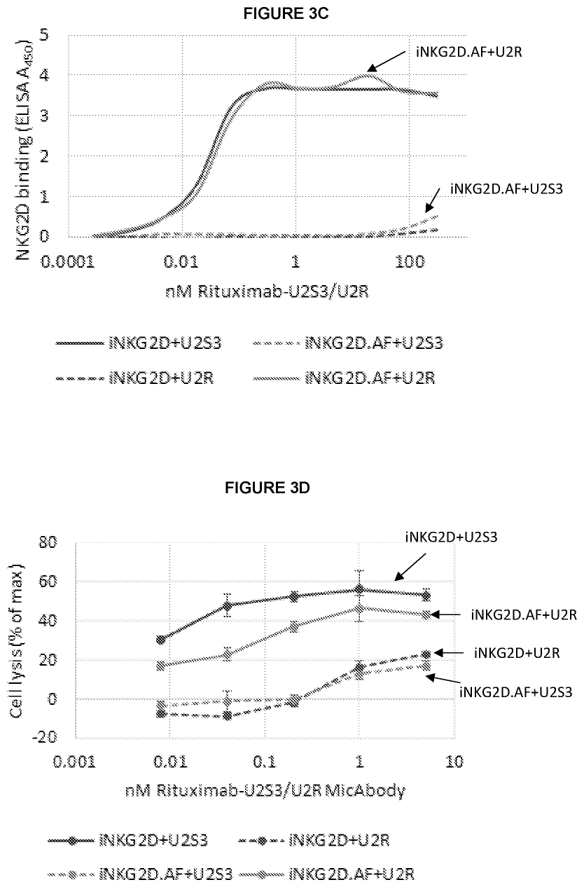

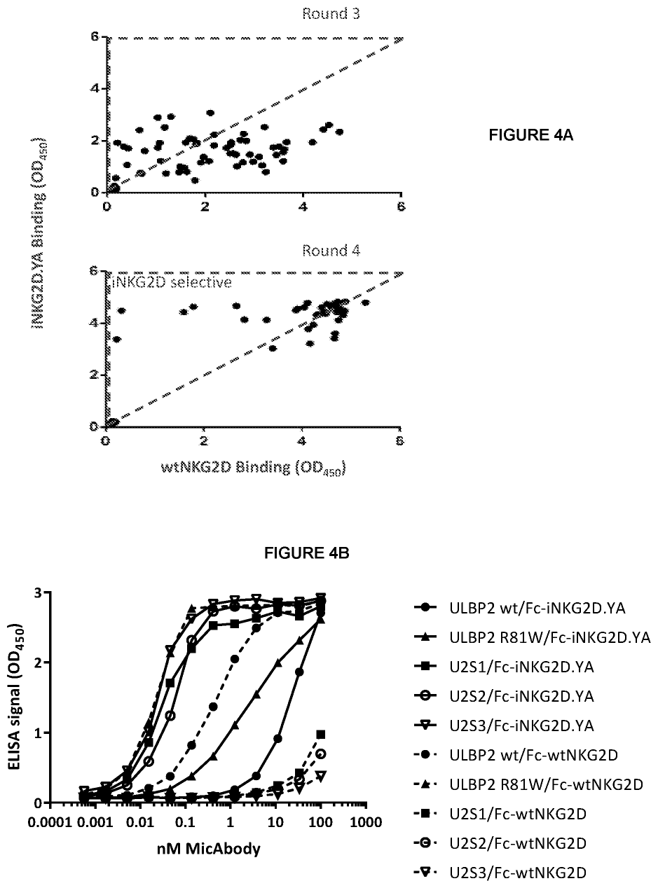

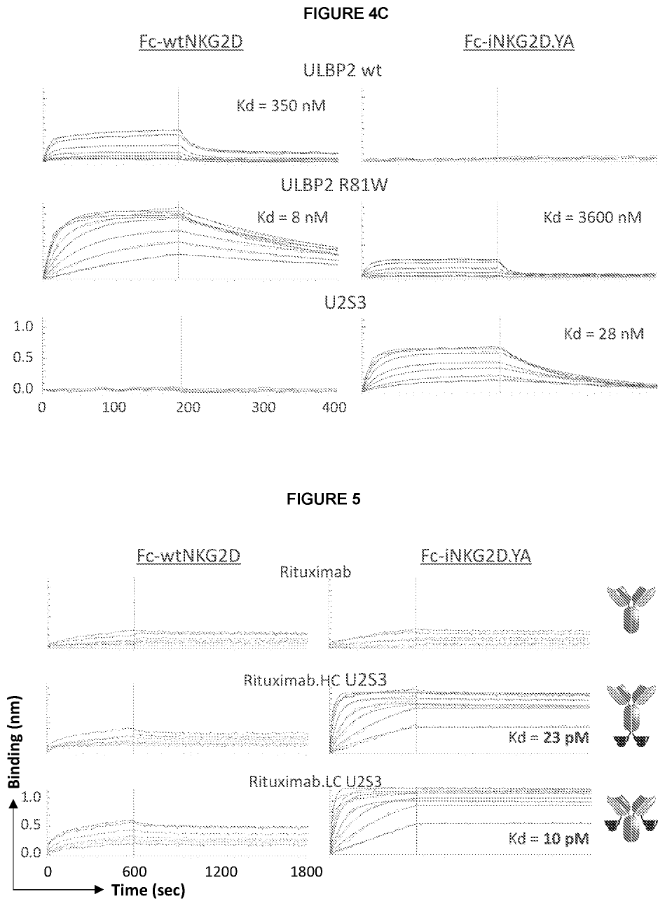

Engineering an orthogonal NKG2D-ligand interaction: Two central tyrosine residues in each NKG2D monomer have critical roles in driving receptor-ligand interactions. Culpepper et al., Mol. Immunol. 48, 516-523 (2011). Mutations at these residues were heavily explored, with the Y152A mutant (“INKG2D.YA”) and the Y152A/Y199F double mutant (“iNKG2D.AF”) selected for further study and confirmed by biolayer interferometry (BLI) (FIGS. 2A and 3A) and ELISA (FIG. 2B) to have lost binding to all naturally occurring human ligands. The ULBP2 A1A2 domain was chosen for phage display-based selection of mutants with high affinity binding to each of the iNKG2D variants since it is not polymorphic. NNK libraries interrogating helix 2 and helix 4 returned only helix 4 variants and even then only in the context of a spontaneous R81W mutation, which likely has a stabilizing role on the ULBP2 A1A2 domain. Competitive selection with rounds of increasing concentration of wtNKG2D (FIG. 4A) yielded three variants-U2S1, U2S2, and U2S3—that reproducibly bound exclusively to iNKG2D.YA, even when reformatted as fusions to the C-terminus of the IgG1 heavy chain of the anti-FGFR3 antibody clone R3Mab (FIG. 4B). Although the R81W mutation alone enhanced affinity towards both wtNKG2D and iNKG2D.YA (FIGS. 4B and 4C), its presence in the INKG2D-selective variants was deemed essential since its reversion to the wild-type residue resulted in loss of binding to iNKG2D.YA. As U2S3 consistently exhibited a greater binding differential, it was characterized more thoroughly and shown as a monomer to have a 10-fold higher affinity towards INKG2D. YA than wild-type ULBP2 had to wtNKG2D (FIG. 4C). The A1-A2 domain of SEQ ID NO: 11 is based on the U2S3 ligand. Picomolar binding to iNKG2D.YA was measured with a bivalent rituximab antibody fusion and orthogonality was retained by both light-chain (LC) and heavy-chain (HC) fusion configurations (FIG. 5).

Candidate orthogonal variants were similarly identified for iNKG2D.AF and ELISAs comparing rituximab-LC fusions to Fc-wtNKG2D, Fc-iNKG2D. YA, and Fc-INKG2D.AF identified four variants that selectively bound only iNKG2D.AF (FIG. 3B) with the U2R variant being the most selective. ELISAs comparing binding of Rituximab.LC-U2S3 and Rituximab.LC-U2R to both INKG2D.YA and INKG2D.AF confirmed that these two independently selected orthogonal ligands exclusively engaged the inert NKG2D variant for which it was evolved (FIG. 3C).

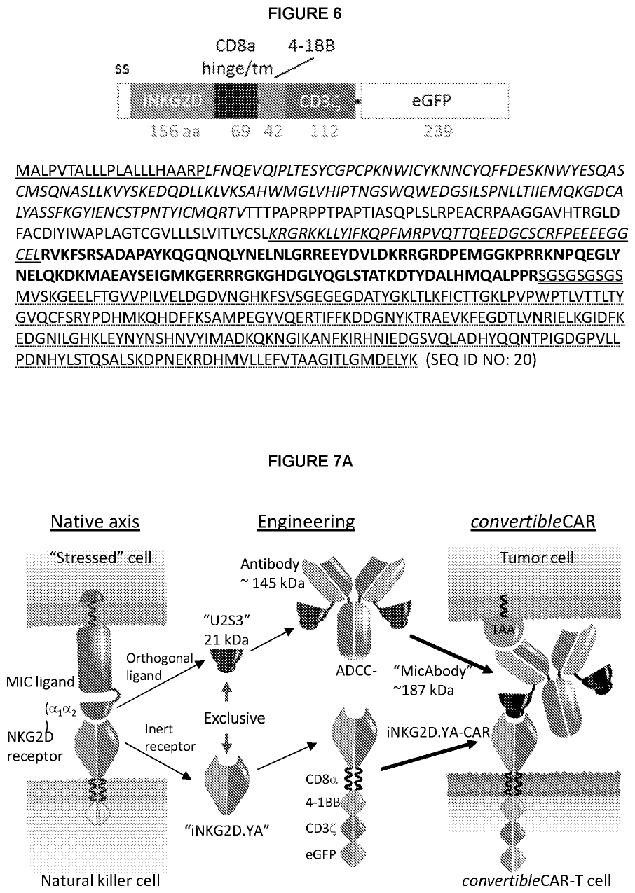

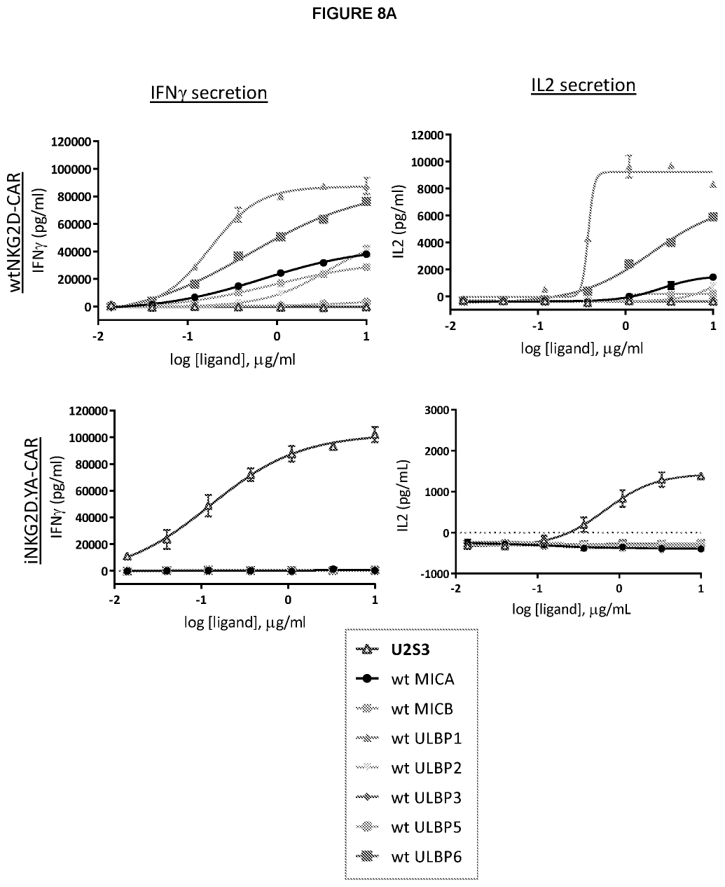

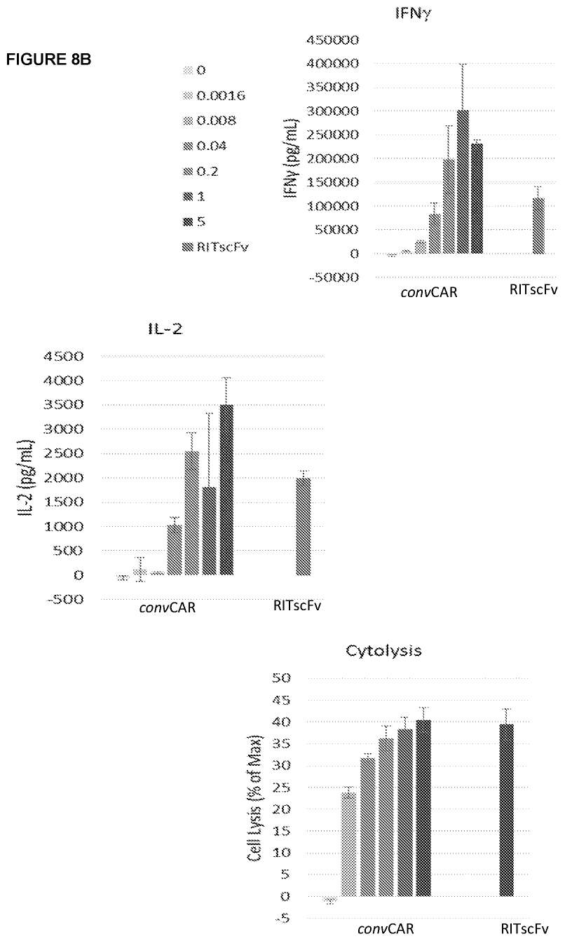

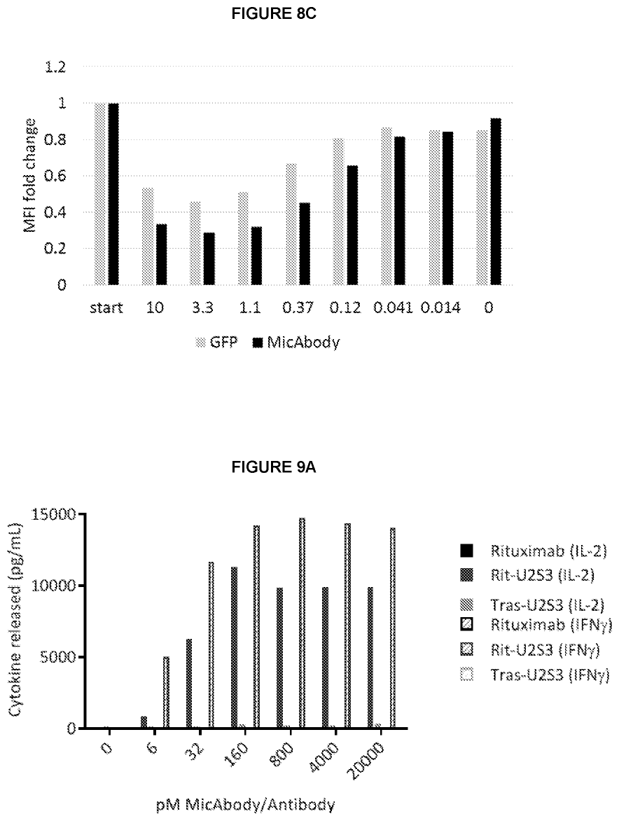

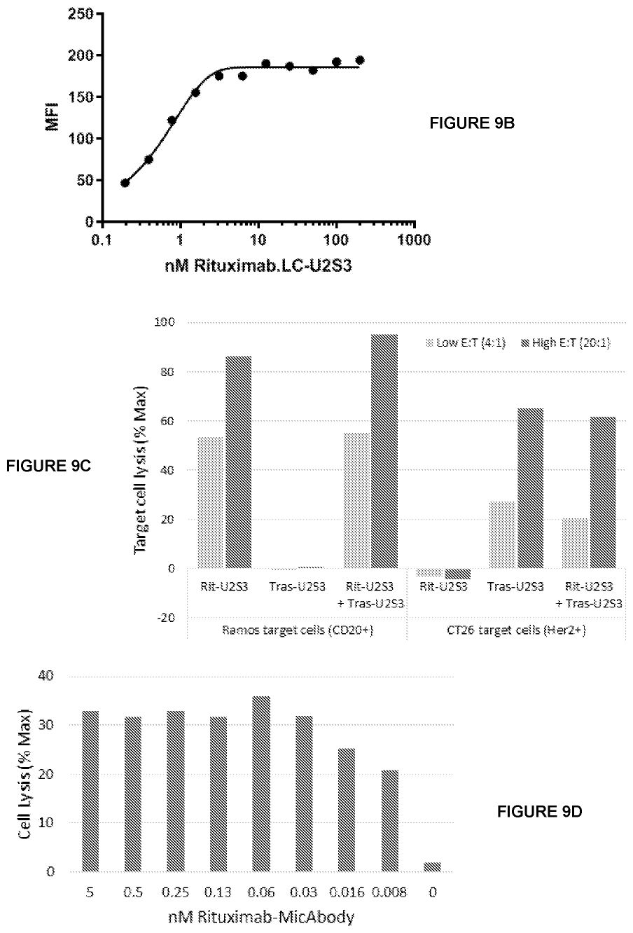

Expression of INKG2D. YA as a chimeric antigen receptor. Lentiviral transduction of iNKG2D.YA fused to 4-1BB, CD3ζ, and eGFP into primary human T cells efficiently generated convertibleCAR-T cells with robust transgene expression on par with a rituximab-scFv based CAR construct (RITscFv-CAR) with the same hinge, transmembrane, and intracellular architecture (FIG. 2b and FIG. 6). Surface staining of INKG2D. YA with the Rituximab.LC-U2S3 MicAbody correlated strongly with GFP expression, suggesting a direct relationship between efficiency of CAR expression and presentation of iNKG2D on the T cell surface (FIG. 7C). Using flow cytometry of Alexa Fluor 647 conjugated Rituximab.LC-U2S3 MicAbody and standard quantification beads, the median amount of iNKG2D. YA expressed on the surface was estimated to be 21,000 molecules. Direct engagement of iNKG2D. YA-CAR receptors by incubation of convertibleCAR-CD8+ cells to microtiter plates coated with wild-type or U2S3 ligands resulted in activation and liberation of IL-2 and IFNγ only with U2S3 while wtNKG2D-CAR bearing cells responded only to wild-type ligands confirming the selectivity of the orthogonal interaction in the context of T cells (FIG. 8A). Furthermore, activation of convertibleCAR-T cell function was dependent upon the presence of the appropriate cognate ULBP2 variant. The INKG2D. YA expressing or iNKG2D.AF expressing T cells only lysed Ramos (CD20+) target cells when armed with a MicAbody bearing its respective orthogonal ligand, i.e. U2S3 or U2R (FIG. 3D). Co-culture of Ramos cells alone was not sufficient to drive activation of convertibleCAR-CD8+ cells. Instead the appropriate antigen-targeting MicAbody was required since neither rituximab antibody nor Trastuzumab.LC-U2S3 activated CAR cells whereas Rituximab.LC-U2S3 triggered maximum cytokine release in the 32-160 pM range. Additionally, cytokine release by convertibleCAR-T cells with Rituximab.LC-U2S3 MicAbody exceeded that of the RITscFv-CAR cells (FIG. 8B). These data demonstrated that the appropriate antigen-targeting MicAbody was required to form a junction, likely similar to that characterized for scFv-CARs19, between the target and convertibleCAR-T cells to drive robust activation of T cell function (FIG. 9A).

Staining of convertibleCAR-CD8+ cells with a fluorescently labeled Rituximab.LC-U2S3 MicAbody revealed saturation of total iNKG2D. YA-CAR receptors at 5 nM (FIG. 9B). However, in a co-culture killing experiment where convertibleCAR-CD8+ cells were armed with decreasing amounts of Rituximab.LC-U2S3, Ramos target cell lysis activity reached a saturation response at 30 PM, two orders of magnitude less than required for full occupancy of receptors (FIG. 9C). This result suggests the extra unoccupied iNKG2D.YA-CAR receptors could be armed with heterologous MicAbodies to guide activity against multiple targets simultaneously. To directly test this, convertibleCAR-CD8+ cells were armed with Rituximab.LC-U2S3, Trastuzumb.LC-U2S3 (targeting Her2), or an equimolar mixture of the two MicAbodies and exposed to either Ramos cells or CT26-Her2. Although CAR cells armed with a single MicAbody directed lysis to only tumor cells expressing the cognate antigen, dual-armed CARs targeted both tumor cell lines without any compromise in lytic potency (FIG. 9D).

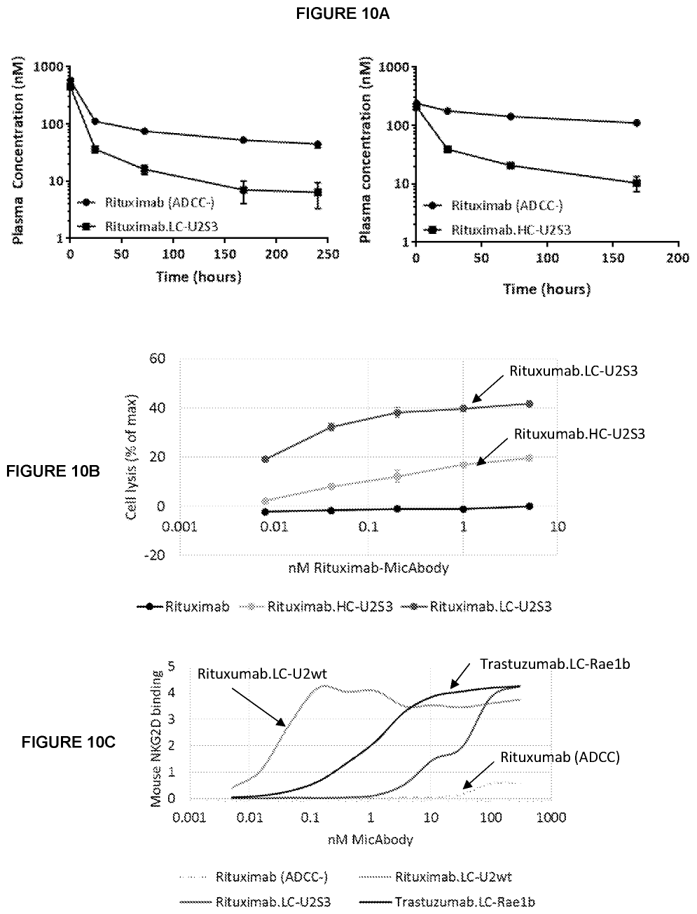

convertibleCAR-T cells inhibit disseminated B-cell lymphomas: The pharmacokinetics of both the HC and LC Rituximab-U2S3 MicAbodies (FIG. 10A) in NSG mice revealed a β-phase that paralleled the parental antibody with the steeper ax-phase of the MicAbodies attributed to retention of U2S3 binding to endogenous mouse wild-type NKG2D (FIG. 10C). The LC-U2S3 fusion (i.e., the antibody fusion protein wherein an A1-A2 domain is fused to the light chain of the antibody) had a longer terminal half-life than the HC-fused MicAbody (i.e., an antibody fusion protein wherein an A1-A2 domain is fused to the heavy chain of the antibody). The LC-U2S3 fusion also out-performed the HC fusion in an in vitro killing assay with Ramos target cells (FIG. 10B), and appeared to be more efficacious at early time points in suppressing Raji B cell lymphoma expansion in NSG mice. In summary, the antibody fusion protein comprising an A1-A2 domain fused to the N-terminus of the light chains of the antibody surprisingly outperformed antibody constructs wherein an A1-A2 domain was fused to the heavy chain.

Rituximab.LC-U2S3 (Rit-S3; the antibody fusion protein wherein the U2S3 A1-A2 domain is fused to the light chain of the antibody) was deployed in further experiments exploring dosing parameters for lymphoma control. An intermediate Rit-S3 dose of 20 μg was shown to be the most efficacious as high concentrations may result in over saturation of receptors on the CAR cells and antigens on the tumor cells, thereby interfering with productive engagement. Additionally, a higher frequency of Rit-S3 administration of every two days versus every four days paired with a higher dose (10×10⁶) of convertibleCAR-T cells resulted in the greatest suppression of tumor growth. Rit-S3 alone was ineffective at tumor control while a graft-vs-tumor effect was consistently observed in both untransduced and convertibleCAR only cohorts. Rit-S3 was detectable in the serum of mice throughout the course of the study with peak levels appearing earlier with more frequent dosing.

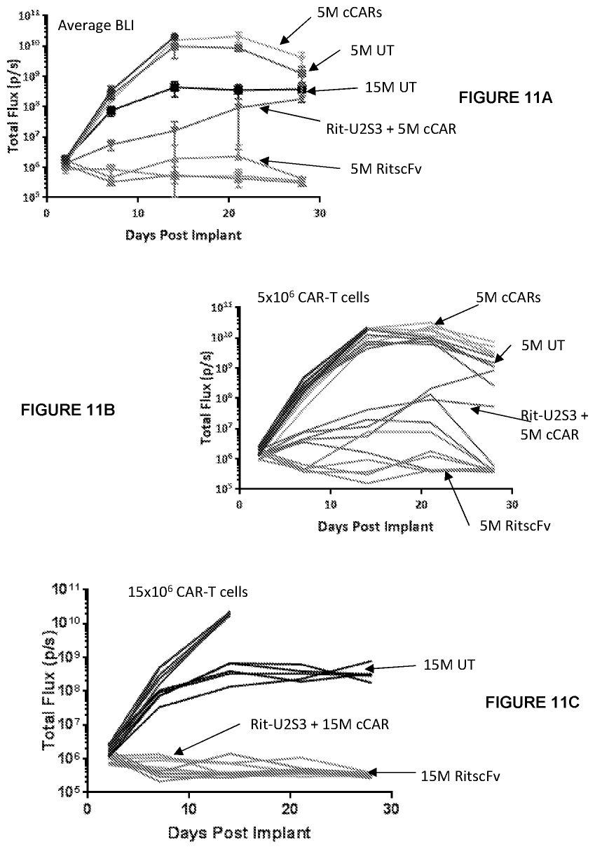

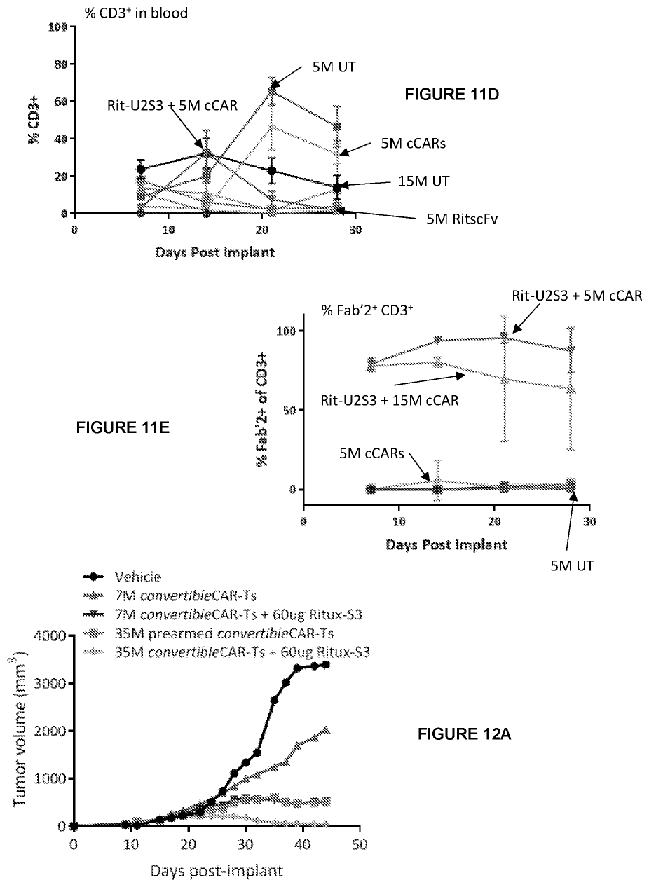

A Raji disseminated lymphoma model with optimized convertibleCAR-T dosing was performed with 20 μg Rit-S3 dosing every two days comparing 5×10⁶ (5M) to 15×10⁶ (15M) convertibleCAR-T cells. As a positive control, RITscFv-CAR cells were also included which have in vitro Ramos killing potency comparable to convertibleCAR-T cells (FIG. 8B). At 5M total T cells, both RITscFv-CAR and convertibleCARs+Rit-S3 were effective at controlling tumor. Although the average tumor bioluminescence signal was lower for the RITscFv-CAR cohort (FIG. 11A), and four of five mice in that cohort appeared to have cleared tumor tissue, three of five mice in the convertibleCAR+Rit-S3 cohort appeared cleared (FIG. 11B). When total infused CAR-T cell doses were increased to 15M cells, both RITscFv-CAR and convertibleCAR+Rit-S3 were able to completely block tumor expansion (FIGS. 11A and 11B). In all studies, peak levels of peripheral human CD3+ T cells consistently appeared around seven days post-infusion with both scFv-CAR and convertibleCAR-T cells having contracted in the majority of mice by 14 days (FIG. 11D). There was a delayed expansion of CD3+ cells in the untransduced and convertibleCAR-only cohorts that was contemporary with the onset of the graft-versus-tumor response and likely the consequence of expansion of specific reactive clones. MicAbody associated convertibleCAR-T cells were observed in the blood of mice in convertibleCAR+Rit-S3 cohorts (FIG. 11E).

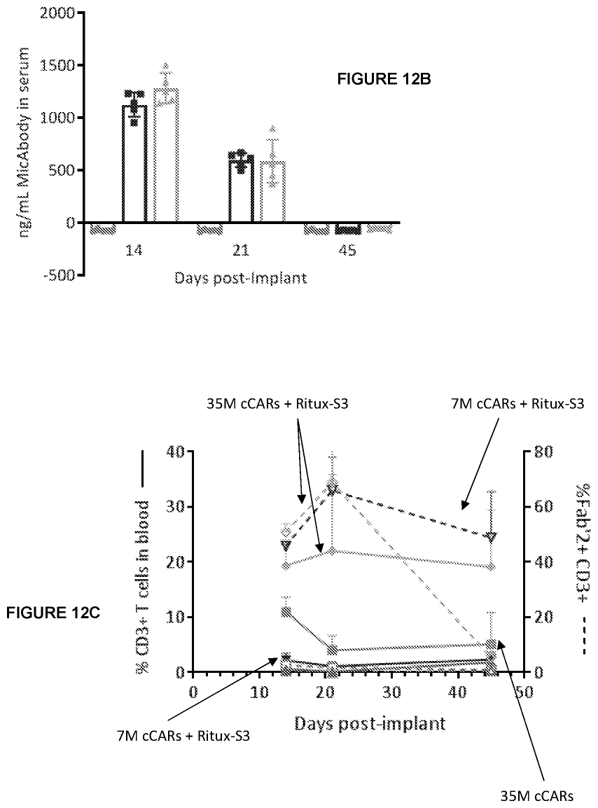

convertibleCAR-T cells inhibit subcutaneous lymphomas: Raji B-cells were implanted subcutaneously to assess the ability of the convertibleCAR system to suppress growth of a solid tumor mass. Once tumors were established at 10 days, either 7×10⁶ (7M) or 35×10⁶ (35M) convertibleCAR-Ts were administered after a single IV dose of 60 μg Rit-S3. Additionally, one cohort received 35M cells that were pre-armed with a saturating concentration of Rit-S3 prior to administration but no additional MicAbody introduced injections. Administration of 7M convertibleCAR-T cells along with Rit-S3 (7M+Rit-S3) resulted in reduced tumor size relative to convertibleCAR-T cells alone (FIG. 12A). Furthermore, in the 35M+Rit-S3 cohort, tumor growth was completely suppressed. Tumor growth in the cohort that received 35M pre-armed cells was also inhibited. By two days post-infusion, convertibleCAR-T cells that had been pre-armed did not have detectable surface associated Rit-S3 MicAbody (FIG. 12C), a result of disarming likely due to a combination of activation-induced cell proliferation and the turnover armed receptors. Serum levels of Rit-S3 were comparable at both CAR-T cell doses across the study and persisted through day 21 when it was detected at approximately 600 ng/ml (3.2 nM) (FIG. 12B) corresponding to high levels of armed peripheral CAR cells (FIG. 12C). By day 45 of the study, the cohort receiving 35M+Rit-S3 maintained relatively high CD3+ T cell numbers but were not well-armed with MicAbody while the 7M+Rit-S3 cohort did have cells that maintained surface-associated MicAbody. This suggested that, as MicAbody levels fell below detectable limits in the plasma, CAR arming could not be maintained at high CAR-T cell levels. An alternative possibility is that the higher CD3+ cell numbers in the 35M+Rit-S3 cohort reflect expansion of a graft-vs-tumor subset of cells that do not express the CAR construct. However, the elevated CD3+ cell numbers were not seen in the 35M pre-armed cohort suggesting that this is not the case. In summary, pre-armed convertibleCAR-Ts were able to exert a potent anti-tumor response that inhibited tumor expansion. Furthermore, convertibleCAR-Ts were able to effectively control a solid lymphoma when an adequate in vivo level of convertibleCAR-T cell arming was maintained.

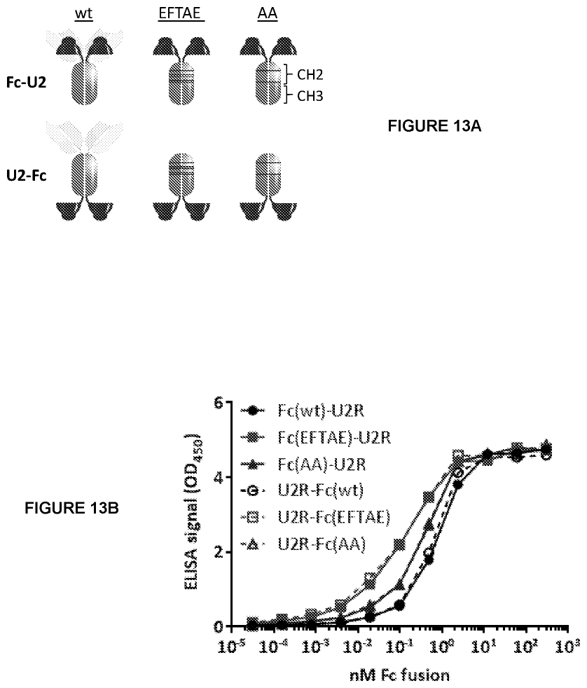

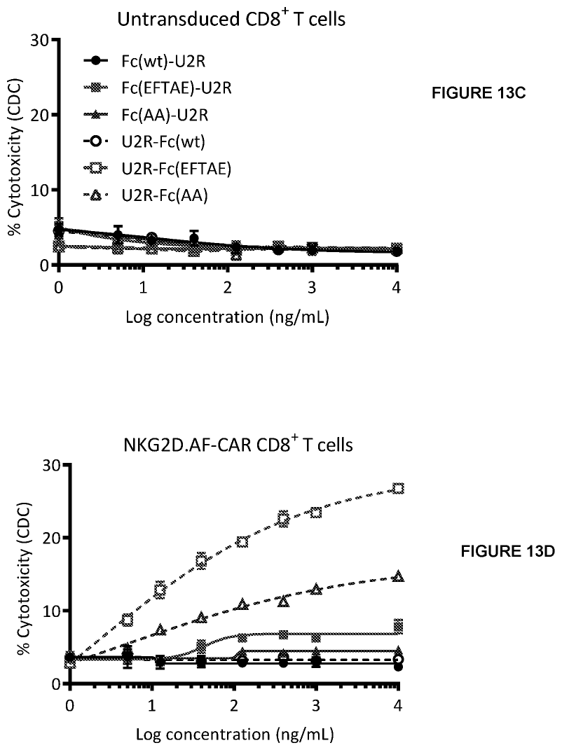

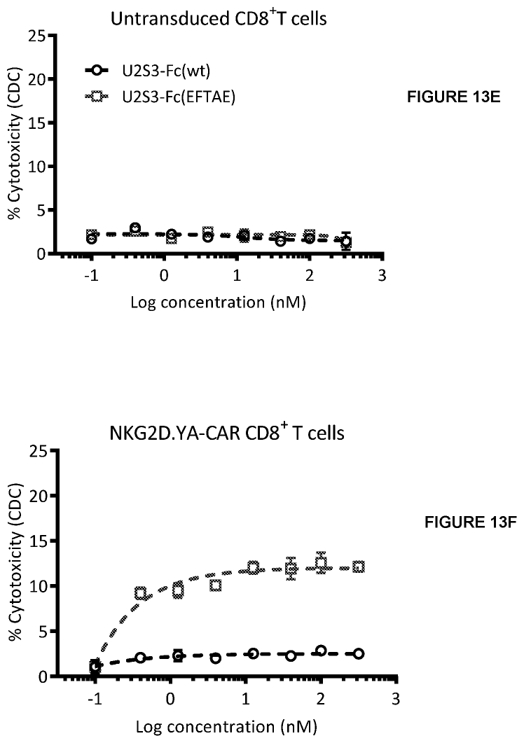

Selective delivery of biomolecules to convertibleCAR-T cells: The privileged interaction between iNKG2D variants and their orthogonal ligands enables the selective delivery of agents to iNKG2D-CAR expressing cells simply by fusing them as payloads to the orthogonal ligands themselves. To demonstrate the utility of this feature two disparate applications were explored: targeted ablation utilizing the complement system and selective delivery of activating cytokines. In the first application, the U2R variant was fused to either the N- or C-terminus of the wild-type human IgG1 Fc-domain or to mutant Fc domains previously described as enhancing C1q binding-S267E/H268F/S324T/G236A/1332E (“EFTAE”)20 and K326A/E333A (“AA”)21 (FIG. 13A). Using just the Fc-portion, as opposed to a complete therapeutic antibody targeting an epitope-tagged CAR cell, avoids collateral effects from opsonization of non-iNKG2D expressing cells. The enhanced C1q binding was confirmed by ELISA with relative order of Kd's as EFTAE<AA<wt (FIG. 13B). While iNKG2D.AF-CAR cells were susceptible to killing by human complement in a manner that was dependent upon both concentration and C1q affinity (FIGS. 13C and 13D), untransduced cells were unaffected. Interestingly, orientation of the U2R fusion was important for function-N-terminal fusions, which orient the Fc in a manner consistent with an antibody, were much more effective. Similar results were obtained with the U2S3 and iNKG2D.YA pairing (FIGS. 13E and 13F).

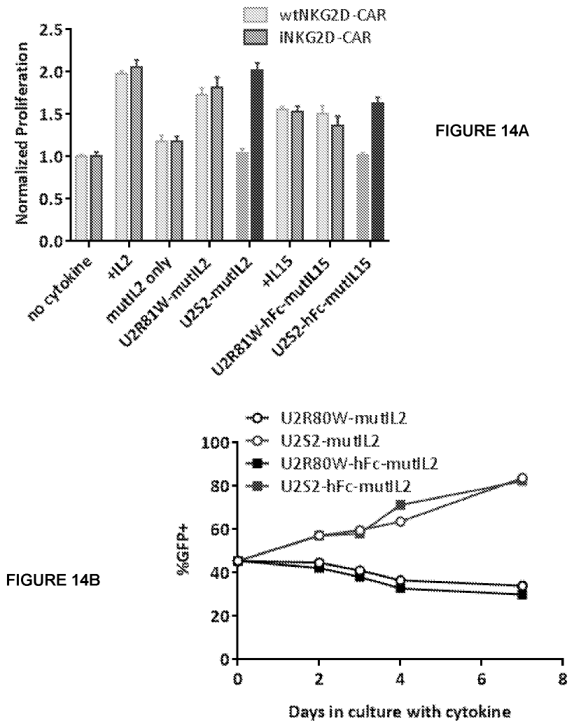

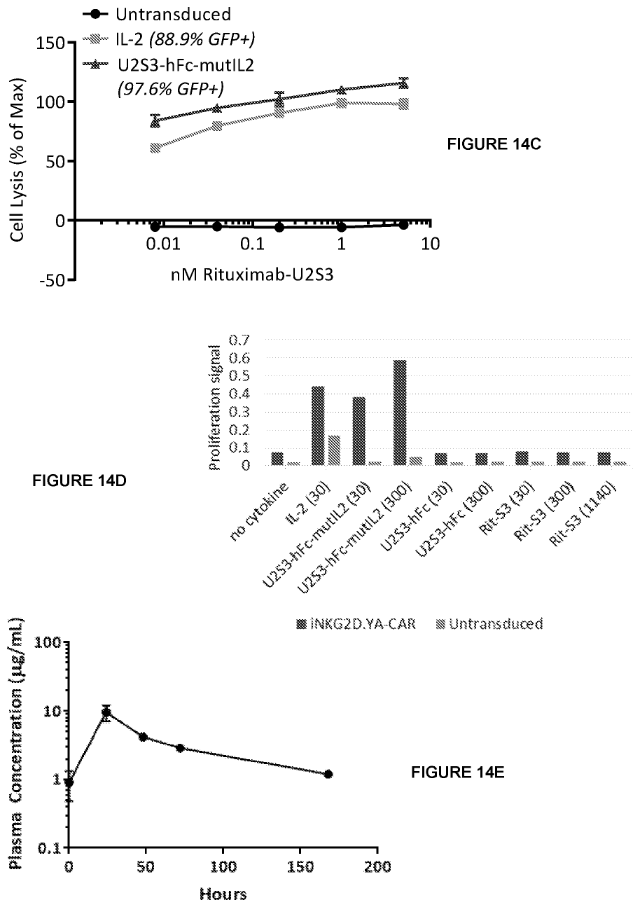

The potential ability of orthogonal ligands to deliver cytokines selectively to iNKG2D-CAR expressing cells has advantages to not only promote their expansion but also potentially leverage differential cytokine signaling to control T cell phenotype and function. As a general design principle, mutant cytokines with reduced binding to their natural receptor complexes were employed to reduce their engagement with immune cells not expressing the CAR and to minimize toxicity associated with wild-type cytokines. Additionally, cytokine fusions were kept monovalent to eliminate avidity-enhanced binding and signaling. To this end, the R38A/F42K mutations in IL-2 (mutIL2)25 and the V49D mutation in IL-15 (mutIL 15) dramatically reduce binding to each cytokine's respective Ra subunit while maintaining IL-2Rβ/γ complex engagement. Initial experiments using the iNKG2D.YA orthogonal variant U2S2 fused to either mutIL2 or mutIL 15 promoted proliferation of INKG2D.YA-CAR expressing cells but not those expressing wtNKG2D-CAR (FIG. 14A). Both cell populations expanded in the presence of the ULBP2.R81W variant, which does not discriminate between wtNKG2D and iNKG2D. YA. Direct fusion to the ligand or via a heterodimeric Fc linkage (e.g., U2S2-hFc-mutIL2) promoted expansion of GFP+ convertibleCAR-T cells to densities above the untransduced cells present (FIG. 14B), and these expanded convertibleCAR-T cells maintained their cytolytic capabilities (FIG. 14C). Engagement of iNKG2D. YA by MicAbody, monovalent U2S3-hFc (without a cytokine payload), or by mutIL2 alone were insufficient to drive proliferation of convertibleCAR-CD8 cells (FIGS. 14A and 14D). Flow cytometry characterization of STAT3 and STAT5 phosphorylation (pSTAT3 and pSTAT5) revealed that exposure to wild-type IL-2 or IL-15 resulted in an increase of pSTAT3 and pSTAT5 in both untransduced as well as convertibleCAR-CD8 cells. Treatment of untransduced cells with U2S3-hFc-mutIL2 resulted only in a minimal shift in pSTAT5 relative to the no cytokine control, consistent with mutIL2's retention of IL-2Rβ/γc binding. The convertibleCAR-CD8 cells responded to both U2S3-hFc-mutIL2 and U2S3-hFc-mutIL 15 with an increase in pSTAT5 levels via γ-chain activation of JAK3. Unlike wild-type cytokines, no increase in pSTAT3 signal was observed, indicating a reduction in JAK1 activation through IL-2Rβ28 in both scenarios as a consequence of disruption of Rα binding, a hypothesis supported by IL-15Rα's role in increasing the affinity of IL-15 for IL-2Rβ. The kinetics of responses U2S3-hFc-mutIL2 and U2S3-hFc-mutIL 15 were nearly identical, indicating functional redundancy in their mutant forms.

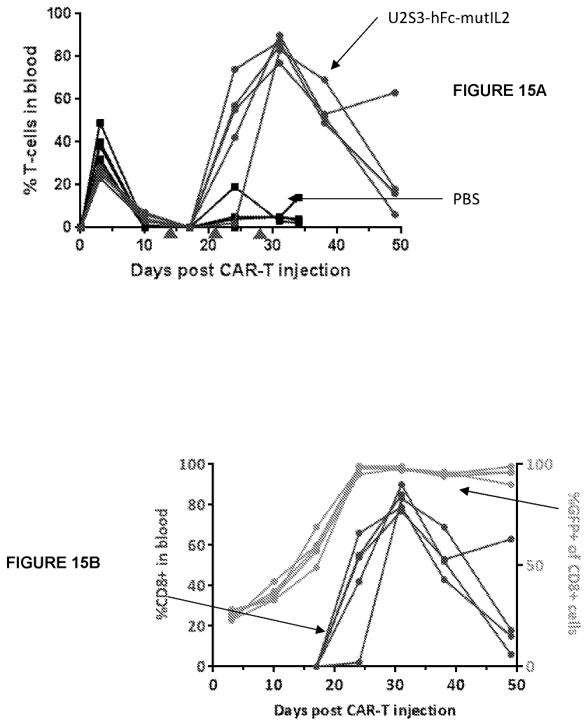

U2S3-hFc-mutIL2 was shown to have an in vivo PK half-life of a few days (FIG. 14E). convertibleCAR-T cells administered to NSG mice in the absence of tumor underwent a homeostatic expansion, peaking at three days followed by contraction. Three injections of U2S3-hFc-mutIL2 staged one week apart resulted in a dramatic expansion of human T cells in the peripheral blood (FIG. 15A) and T cell numbers contracted after cessation of U2S3-hFc-mutIL2 support with CD8+ T cells driving the bulk of the expansion. In parallel with expansion, the proportion of GFP+CD8+ T cells increased to 100% demonstrating selective expansion of iNKG2D-CAR expressing cells but not untransduced cells (FIG. 15B).

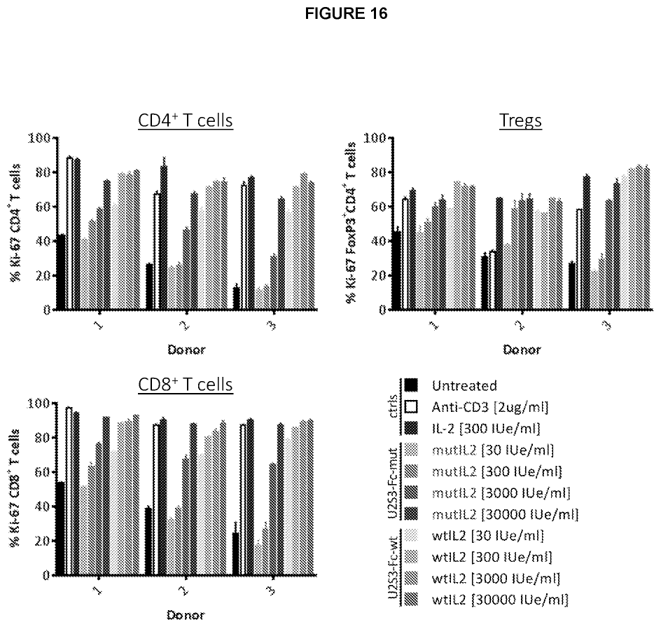

The effect of U2S3-hFc-mutIL2 on normal human PBMCs from three donors was explored in vitro by exposure to increasing concentrations of the agent for four days followed by flow-based quantification of cells positive for the proliferative marker Ki-67 (FIG. 16). In addition to the -mutIL2 fusion, a wild-type IL2 fusion (U2S3-hFc-wtIL2) was included to directly demonstrate that the reduction in mutIL2 bioactivity was a consequence of the mutations employed and not the fusion format itself. The CD4+ and CD8+ T cells responded robustly to both anti-CD3 and wild-type IL-2 positive controls as well as to the lowest dose of U2S3-hFc-wtIL2. Proliferative responses to U2S3-hFc-mutIL2 occurred in a dose-dependent manner with expansion observed across donors at levels above 300 IUe/mL but not achieving levels comparable to those of the IL-2 positive control until 30,000 IUe/mL. Treg responses were comparable to those of CD4+ and CD8+ cells with the exception of cells from one donor (who additionally had a muted response to anti-CD3 stimulation) that responded to U2S3-hFc-mutIL2 at a lower concentration than the other donors. Taken together, these data support the hypothesis that normal human PBMCs do not respond to U2S3-hFc-mutIL2 except at super-physiologic levels which potentially provides a wide dosing window for selective delivery of ligand-fused mutIL2 to convertibleCAR cells while minimizing toxicity and Treg activation.

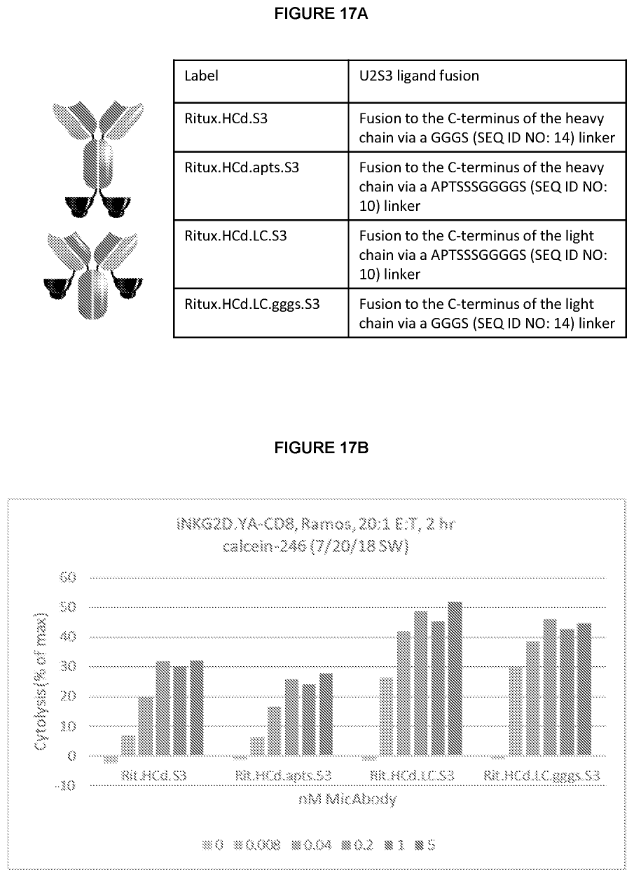

Comparison of A1-A2 domain location and linkers: In addition to the studies above, constructs comprising different linkers connecting the antibody heavy or light chains to A1-A2 domains were studied. See FIG. 17A. Rituximab antibodies comprising a U2S3-based A1-A2 domain attached to the heavy chains (Ritux.HCd) via a GGGS (SEQ ID NO: 14) linker (Ritux.HCd.S3) or an APTSSSGGGS (SEQ ID NO: 10) linker (Ritux.HCd.apts.S3) were generated. Similar constructs were generated where A1-A2 domains were fused to the light chains of the Rituximab antibody via the same linkers (Ritux.HCd.LC.S3 (APTSSSGGGGS linker (SEQ ID NO: 10)) and Ritux.HCd.LOC.gggs.S3 (GGGS linker (SEQ ID NO: 14))). The constructs were examined using the methods described herein in connection with FIG. 10B. The results are illustrated in FIG. 17B. Antibody fusion constructs comprising heavy chains comprising variable region sequences of SEQ ID NO: 1 and light chains comprising variable region sequences of SEQ ID NO: 8, wherein the light chains were fused at the C-terminus to an A1-A2 domain, outperformed constructs wherein the A1-A2 domain was attached to heavy chains in killing tumor cells. Additionally, constructs wherein the A1-A2 domain were fused to the light chains via the APTSSSGGGGS linker (SEQ ID NO: 10) surprisingly outperformed all constructs tested at almost all concentrations (0.04 nM, 0.2 nM, 1 nM, and 5 nM).

### Discussion

The disclosure describes the engineering of a privileged receptor-ligand (iNKG2D.YA and U2S3) pairing comprised of human components for a highly adaptable CAR, resulting in a versatile and broadly controllable platform. The iNKG2D.YA-CAR receptor itself is held invariant on T cells with CAR function readily directed to potentially any antigen of interest by virtue of attaching the orthogonal ligand to the appropriate antigen-recognizing antibody. In this manner, the same convertibleCAR-T cells can be retargeted as needed if, for example, the original tumor antigen becomes downregulated during the course of therapy. This targeting flexibility is not limited to sequential engagement of antigens, but can also be multiplexed to simultaneously direct T cells to more than one antigen in order to reduce the likelihood of tumor escape by antigen loss, address the issue of heterogeneity of intratumoral antigen expression, or even simultaneously target tumor and suppressive cellular components of the tumor microenvironment. Traditional scFv-CAR cells are generally committed to a fixed expression level of a receptor which reduces their ability to discriminate between antigen levels present on healthy versus aberrant cells. The use of switch/adaptor strategies, like MicAbodies with convertibleCAR-T cells, may provide an opportunity to differentially engage CAR-Ts to achieve a therapeutic index that reduces the risk of severe adverse events.

The use of the privileged receptor-ligand interaction for delivery of payloads specifically to iNKG2D-bearing cells without additional cellular engineering is another advantage. The capability of harnessing interleukin functions to drive expansion and activation, prevent exhaustion, or even promote suppression in a controlled and targeted manner could have beneficial consequences for efficacy and safety. Introduction of cytokine-ligand fusions during CAR manufacturing could address qualitative and quantitative limitations of patient T cells and their administration post-CAR infusion could expand the number of CAR-T cells and their persistence which, with CD19-CAR therapies, is correlated positively with response rates. Most CAR therapies require a preconditioning lymphodepletion regimen to promote engraftment and expansion of CAR cells, one rationale being that it provides a more verdant immunological setting for CARs to expand. Robust and controllable convertibleCAR-T expansion in patients may supplant the need for lymphodepletion, allowing for retention of endogenous immune functions that are fully competent to support the initial convertibleCAR-mediated anti-tumor activity. Another clinical strategy might be to deliver cytokine-ligand fusions to bolster convertibleCAR-T function, possibly with a cycling regimen to reduce T cell exhaustion and promote the maintenance of memory T cells. And lastly, as CARs have been demonstrated to persist in humans for years post-infusion, the ability to recall resident convertibleCAR-Ts to attack primary or secondary malignancies (either with the original targeting MicAbody or a different one) without having to re-engineer or generate a new batch of CAR cells should be highly advantageous. Unlike scenarios where CARs have been engineered to constitutively express cytokines, delivery of cytokines exclusively to convertibleCAR-T cells can be modulated depending upon the manufacturing or clinical needs.

By design, each component of the convertibleCAR system—the iNKG2D-based CAR receptor and the MicAbody (which is ADCC-deficient)—are functionally inert on their own. This has advantages during manufacturing, particularly in the context of indications such as T cell malignancies where traditional scFv-based CARs encounter expansion hurdles due to fratricide. Additionally, it provides enhanced control of CAR function during treatment. The disclosure demonstrates that convertibleCAR-T cells can be armed with MicAbody prior to administration to provide an initial burst of anti-tumor activity on par with traditional scFv-CARs. In addition to activation-induced replication, these cells also internalize their engaged CAR receptors in a manner consistent with what has been observed with other 4-1BB/CD3zeta scFv-CARs. As a consequence of these two processes, convertibleCAR-T cells will rapidly disarm after initial expansion and target engagement, which then provides an opportunity rearm and re-engage in a manner controlled by MicAbody dosing.

In addition to the iNKG2D-U2S3 pairing based upon ULBP2, the disclosure identifies high-affinity orthogonal MicA and ULBP3 variants to iNKG2D.YA that are non-redundant in their amino acid compositions through the helix 4 domain. Additionally, a completely independent iNKG2D.AF and U2R pairing is described. Having mutually exclusive receptor-ligand pairs enables, for example, their introduction into distinct cell populations (e.g., CD4 and CD8 T-cells) to differentially engage them as needed. Furthermore, within the same cell, the two iNKG2D variants could be expressed with split intracellular signaling domains to provide dual antigen-dependent activation to enhance on-tumor selectivity. Alternatively, the two iNKG2D variants could be differentially linked to either activating or immunosuppressive domains to enhance the discriminatory power of the T cells between tumors or healthy tissue, respectively.

In summary, the system described herein has demonstrated capabilities to not only be readily targeted to different cell-surface antigens but can also be selectively engaged exogenously to drive cell expansion. The privileged receptor-ligand interaction that has been developed is agnostic to cell type and can be engineered into any cell of interest as long as the cell-appropriate signaling domains are provided. Additionally, the adoptive cellular therapy field is aggressively pursuing the development of allogeneic cells to bring down the time, complexity, and cost of manufacturing to provide a more consistent, readily accessible product. A highly adaptable CAR system would be powerfully synergistic with allogenic efforts and once a truly universal allogeneic CAR system has been validated, the therapeutic field then becomes characterized by the relative ease of developing and implementing a library of adaptor molecules from which personalized selections can be made. This strategy also broadens the potential areas of application to any pathogenic call with a targetable surface antigen.

All references, including publications, patent applications, and patents, cited herein are hereby incorporated by reference to the same extent as if each reference were individually and specifically indicated to be incorporated by reference and were set forth in its entirety herein.

What is claimed:

1. An antibody fusion protein comprising (i) heavy chains comprising variable region sequences comprising the amino acid sequence of SEQ ID NO: 1 and (ii) light chains comprising variable region sequences comprising the amino acid sequence of SEQ ID NO: 8, wherein the light chains are fused at the C-terminus to an A1-A2 domain comprising the amino acid sequence of SEQ ID NO: 11.

2. The antibody fusion protein of claim 1, wherein the A1-A2 domains are fused to the light chains via a linker comprising the amino acid sequence of SEQ ID NO: 10

3. The antibody fusion protein of claim 1 or claim 2, comprising light chains comprising the amino acid sequence of SEQ ID NO: 13.

4. The antibody fusion protein of any one of claims 1-3, wherein the heavy chains comprise constant domains comprising the amino acid sequence of SEQ ID NO: 3.

5. The antibody fusion protein of claim 4, comprising heavy chains comprising the amino acid sequence of SEQ ID NO: 7.

6. The antibody fusion protein of any one of claims 1-3, wherein the heavy chains comprise constant domains comprising the amino acid sequence of SEQ ID NO: 2.

7. The antibody fusion protein of claim 6, comprising heavy chains comprising the amino acid sequence of SEQ ID NO: 6.

8. An antibody fusion protein comprising light chains comprising the amino acid sequence of SEQ ID NO: 13 and heavy chains comprising the amino acid sequence of SEQ ID NO: 7.

9. An antibody fusion protein comprising light chains comprising the amino acid sequence of SEQ ID NO: 13 and heavy chains comprising the amino acid sequence of SEQ ID NO: 6.

10. A nucleic acid molecule comprising a nucleotide sequence encoding the light chain of the antibody fusion protein of any one of claims 1-9.

11. A composition comprising the nucleic acid molecule of claim 10 and a nucleic acid molecule comprising a nucleotide sequence encoding the heavy chain of the antibody fusion protein of any one of claims 1-9.

12. An expression vector comprising the nucleic acid molecule of claim 10.

13. The expression vector of claim 12, further comprising a nucleic acid molecule comprising a nucleotide sequence encoding the heavy chain of the antibody fusion protein of any one of claims 1-9.

14. A host cell comprising the expression vector of claim 12 or claim 13.

15. A host cell comprising the expression vector of claim 12 and an expression vector comprising a nucleic acid molecule comprising a nucleotide sequence encoding the heavy chain of the antibody fusion protein of any one of claims 1-9.

16. A method of producing an antibody fusion protein, the method comprising
culturing a host cell comprising a nucleic acid molecule comprising a nucleotide sequence encoding the light chain of the antibody fusion protein of any one of claims 1-9 and a nucleic acid molecule comprising a nucleotide sequence encoding the heavy chain of the antibody fusion protein of any one of claims 1-9, and
recovering said antibody fusion protein.

17. A kit comprising one or more containers comprising the antibody fusion protein of any one of claims 1-9 and instructions for use.

18. The kit of claim 17, further comprising one or more containers comprising a mammalian cell comprising a chimeric antigen receptor comprising SEQ ID NO: 15.

19. The kit of claim 18, wherein the mammalian cell is a human lymphocyte or a human macrophage.

20. The kit of claim 18 or claim 19, wherein the chimeric antigen receptor further comprises SEQ ID NOs: 16-18.

21. A method of treating a subject suffering from a CD20-positive cancer, the method comprising administering to the subject the antibody fusion protein of any one of claim 1-9 and a mammalian cell comprising a chimeric antigen receptor comprising SEQ ID NO: 15.

22. The method of claim 21, wherein the mammalian cell is a human lymphocyte or a human macrophage.

23. The method of claim 21 or claim 22, wherein the chimeric antigen receptor further comprises SEQ ID NOs: 16-18.
